(19) United States
(12) Patent Application Publication
(10) Pub. No.: US 2023/0358726 A1
(43) Pub. Date: Nov. 9, 2023

(54) NON-INVASIVE FUNCTIONAL COMPANION ASSAYS FOR ONCOGENE TARGETED THERAPY FOR BRAIN CANCER

(71) Applicant: The Regents of the University of California, Oakland, CA (US)

(72) Inventors: David A. Nathanson, Los Angeles, CA (US); Jonathan Tsang Tsang, Los Angeles, CA (US); Timothy F. Cloughesy, Calabasas, CA (US); Wilson X. Mai, Sunnyvale, CA (US); Benjamin M. Ellingson, Los Angeles, CA (US)

(21) Appl. No.: 18/027,311

(22) PCT Filed: Sep. 21, 2021
(86) PCT No.: PCT/US21/51338

§ 371 Date: Mar. 20, 2023

Related U.S. Application Data
(60) Provisional application No. 63/081,217, filed on Sep. 21, 2020.

(51) Int. Cl. G01N 33/50; A61K 31/517; A61K 51/04; A61B 6/03; A61P 35/00

(52) U.S. Cl. CPC G01N 33/5008; A61K 31/517; A61K 51/0491; A61B 6/037; A61P 35/00; G01N 2800/52; G01N 2800/7071

(57) ABSTRACT

The present disclosure relates to methods for determining the metabolic responder status of a subject having glioblastoma (GBM) and adjusting the course of treatment accordingly. The disclosure further relates to methods of treating GBM and other EGFR-mediated cancers. The disclosure further relates to methods of identifying effective treatments for subjects having GBM.

### CROSS-REFERENCE TO RELATED APPLICATION

This application claims the benefit of U.S. Provisional Application No. 63/081,217, filed Sep. 21, 2020, the entire contents of which are incorporated herein by reference.

### STATEMENT OF GOVERNMENT SUPPORT

This invention was made with government support under Grant Numbers CA211015 and CA213133, awarded by the National Institutes of Health. The government has certain rights in the invention.

### BACKGROUND

Glioblastoma (glioblastoma multiforme; GBM) accounts for most primary malignant brain tumors in adults Amplification and mutation of the epidermal growth factor receptor (EGFR) gene is a signature genetic abnormality encountered in GBM (Sugawa, et al. (1990) Proc. Natl. Acad. Sci. 87: 8602-8606; Ekstrand, et al. (1992) Proc. Natl. Acad. Sci. 89: 4309-4313). A range of potential therapies that target EGFR or its mutant constitutively active form, ΔEGFR, including tyrosine kinase inhibitors (TKIs), monoclonal antibodies, vaccines, and RNA-based agents, are currently in development or in clinical trials for the treatment of GBM. However, to date their efficacy in the clinic has so far been limited by both upfront and acquired drug resistance (Taylor, et al. (2012) Curr. Cancer Drug Targets. 12:197-209). A major limitation is that current therapies such as erlotinib, lapatinib, gefitinib and afatinib are poorly brain penetrant (Razier, et al. (2010) Neuro-Oncology 12:95-103; Reardon, et al. (2015) Neuro-Oncology 17:430-439; Thiessen, et al. (2010) Cancer Chemother. Pharmacol. 65:353-361). Another limitation is that some subjects may not respond to therapy administered. The presence of a specific genetic alteration is often insufficient to predict efficacy to targeted therapies. Thus, subjects can often be given a drug that will ultimately be ineffective. This can lead to lost time that could have been used to treat these subjects with a more effective drug, while also incurring significant costs due to the considerable expense associated with the treatment of a particular targeted agent. In GBM, this issue is compounded by the fact that the short-term action of a particular drug on the tumor (e.g., drug-target engagement) cannot be determined as repeated surgical sampling is impractical.

In view of the foregoing, there is an urgent need for non-invasive techniques to assess the treatment of glioblastoma and other cancers.

### SUMMARY

The present embodiments are directed to methods of determining if a subject will respond to a treatment for glioblastoma. More specifically, the methods disclosed herein relate to identifying or selecting subjects that are responsive to EGFR inhibitors.

### BRIEF DESCRIPTION OF THE DRAWINGS

FIG. 1 is a graph showing the growth inhibition of primary glioblastoma multiforme (GBM) cells treated with erlotinib. The graph illustrates that genetic alterations (e.g., EGFR amp/mutation/CN gain (polysomy) cannot always predict response to targeted therapy.

FIGS. 2A and 2B illustrate that a subset of GBM cells termed “metabolic responders” show rapid reduction in glucose uptake following EGFR TKI treatment. FIG. 2A is an illustration showing how GBM cells obtained from a brain biopsy are cultured to form gliomaspheres. FIG. 2B is a graph showing the change in ¹⁸F-FDG PET uptake after treatment with erlotinib.

FIGS. 3A and 3B illustrate that lactate secretion and extracellular acidification rate (i.e., changes in pH of the media) are linked to a metabolic response, and therefore the treatment response, in vitro. FIG. 3A is a graph showing lactate secretion in multiple responder and non-responder samples in vitro. FIG. 3B is a graph showing the extracellular acidification rate in responder and non-responder samples in vitro.

FIGS. 4A-4D illustrate that metabolic responders have greater attenuation of downstream signaling following EGFR TKI relative to non-responders. FIG. 4A is a graph comparing EGFR activation between responders and non-responders. FIG. 4B is a graph comparing MAPK signaling between responders and non-responders. FIG. 4C is a graph showing AKT signaling between responders and non-responders. FIG. 4D is a graph showing mTOR signaling between responders and non-responders.

FIGS. 5A, 5B, and 5C illustrate that EGFR TKI reduces growth and viability only in metabolic responders. FIG. 5A is a diagram of assessing metabolic responding and nonresponding cells 72 hours after erlotinib treatment. FIG. 5B is a graph showing the fold change in cell number in metabolic responders and non-responders with and without erlotinib treatment. FIG. 5C is a graph showing the percentage of metabolic responder and non-responder cells undergoing apoptosis after treatment with erlotinib.

FIG. 6 comprises a heatmap of exosome sequencing results of metabolic responders and non-responders, which shows that EGFR “alterations” (i.e. amp/CN gain/mutation) are not sufficient to predict a metabolic response.

FIGS. 7A-7G illustrate that ¹⁸F-FDG PET rapidly (i.e., within hours) predicts therapeutic outcome in vivo. FIG. 7A is a diagram showing the experimental setup to identify responders and non-responders to erlotinib. FIG. 7B comprises ¹⁸F-FDG PET scans of responder mice before and after erlotinib treatment. FIG. 7C is a graph showing the reduction of ¹⁸F-FDG uptake over 24 hours in responder mice. FIG. 7D is a graph showing the relative tumor volume in responder mice treated with erlotinib or vehicle. FIG. 7E comprises ¹⁸F-FDG PET scans of non-responder mice before and after erlotinib treatment. FIG. 7F is a graph showing the reduction in ¹⁸F-FDG uptake over 24 hours in non-responder mice. FIG. 7G is a graph showing the relative tumor volume in non-responder mice treated with erlotinib or vehicle.

FIGS. 8A-8C illustrate that JCNO68, like other EGFR TKIs, rapidly inhibits glucose metabolism specifically in GBM cells. FIG. 8A is a graph showing the change in glucose uptake relative to control in GBM39 cells (EGFRvIII) treated with Erlotinib, Lapatinib, or JCNO68. FIG. 8B is a graph showing the change in glucose uptake relative to control in GS025 cells (EGFR amp) treated with Erlotinib, Lapatinib, or JCNO68. FIG. 8C is a graph showing the change in glucose uptake relative to control in normal human astrocytes (NHA) treated with Erlotinib, Lapatinib, or JCNO68.

FIGS. 9A-9C illustrate that rapid changes in glucose metabolism with JCNO68 is associated with response (i.e., low GI50). FIG. 9A is a graph showing the GI50 for GBM39 cells (EGFRvIII) treated with Erlotinib, Lapatinib, or JCNO68. As used in these figures, “EFGRi” denotes EGFR inhibitors. FIG. 9B is a graph showing the GI50 for GS025 cells (EGFR amp) treated with Erlotinib, Lapatinib, or JCNO68. FIG. 9C is a graph comparing the GI50 percentage observed in normal human astrocytes (NHA) and GBM cells.

FIGS. 10A and 10B illustrate that brain penetrant JCNO68, but not the brain impenetrant erlotinib, rapidly decreases ¹⁸F-FDG uptake in intracranial GBM. FIG. 10A comprises ¹⁸F-FDG PET scans of a GBM before and 72 hours after treatment with erlotinib and a graph showing no change in the survival curve between GBM treated with erlotinib and GBM treated with vehicle. FIG. 10B comprises ¹⁸F-FDG PET scans of a GBM before and 72 hours after treatment with JCNO68 and a graph showing increased survival of GBM treated with JCNO68 relative to GBM treated with vehicle.

FIGS. 11A and 11B illustrate that ¹⁸F-FDG PET scans detect metabolic responders to JCNO68. FIG. 11A comprises ¹⁸F-FDG PET scans of a GBX301 (EGFR/EFGRvIII) mouse before and 72 hours after treatment with JCN068 and a graph showing increased survival of the GBX301 mouse treated with JCN068 relative to GBX301 mice treated with vehicle. FIG. 11B comprises ¹⁸F-FDG PET scans of a GBX336 (EGFR Polysomy) mouse before and 72 hours after treatment with JCNO68 and a graph showing increased survival of the GBX336 mice treated with JCNO68 relative to GBX336 mice treated with vehicle.

FIGS. 12A and 12B illustrates that JCNO68 is ineffective in tumors in which the drug does not decrease ¹⁸F-FDG uptake. FIG. 12A comprises ¹⁸F-FDG PET scans of a GBX054 (EGFR wild type) mouse before and 72 hours after treatment with JCNO68 and a graph showing no difference in tumor growth between the GBX054 mice treated with JCNO68 relative to GBX054 mice treated with vehicle. FIG. 12B comprises ¹⁸F-FDG PET scans of a GBX027 (EGFR Polysomy) mouse before and 72 hours after treatment with JCNO68 and a graph showing no difference in tumor growth between the GBX336 mice treated with JCNO68 relative to GBX336 mice treated with vehicle.

FIG. 13 is a graph showing the survival benefit of FDG responders and non-responders.

FIGS. 14A and 14B illustrate that extracellular acidification rate (i.e., changes in pH of the media) are linked to a metabolic response, and therefore the treatment response, in vivo. FIG. 14A is a schematic of the experimental design used to determine the extracellular acidification rate (ECAR) in control and treated GBM grafts. FIG. 14B is a graph showing the extracellular acidification rate in responder and control samples in vitro.

FIGS. 15A-15C show the study design for using ¹⁸F-FDG PET to detect responders in patient derived xenographs (PDXs). FIG. 15A illustrates that the sources of PDXs could be grouped according to the alterations, if any, present in the EGFR gene or its expression. FIG. 15B is a timeline showing that three consecutive measurements of tumor growth are necessary prior to administering treatment to the subjects. FIG. 15C is an illustration of the treatments to be provided, measurements to be taken, and the outcomes to be assessed.

FIGS. 16A and 16B show the overall survival response rate in all mice tested. FIG. 16A is a pie chart showing that over 50% of all treated showed a survival benefit relative to mice that were administered a vehicle control. FIG. 16B shows that the survival benefit ranged from about 50% to about 200%.

FIG. 17 comprises pie charts showing the survival benefits observed from mice with PDXs from glioblastomas having an EGFR mutation, amplified EGFR, or EGFR polysomy.

FIGS. 18A and 18B show ¹⁸F-FDG PET are representative images of glioblastoma PDXs that are metabolic inhibitor responders or non-responders. FIG. 18A is an image showing a metabolic inhibitor responsive glioblastoma PDX. FIG. 18B is an image of a glioblastoma PDX that is non-responsive to a metabolic inhibitor.

FIGS. 19A and 19B show the survival response rate in mice having PDXs derived from glioblastomas having EGFR polysomy. FIG. 16A comprises pie charts showing that of the PDXs that were responsive to a metabolic inhibitor (JCNO68), 80% of the mice showed a survival benefit, but the non-responders showed no survival benefit. FIG. 16B shows that the survival benefit ranged from about 50% to about 200%.

### DETAILED DESCRIPTION

The present disclosure is based, at least in part, on the discovery that rapid changes in glucose metabolism following treatment with a brain penetrant EGFR TKI can predict its therapeutic efficacy in GBM.

Gliomas are the most commonly occurring form of brain tumor, with glioblastoma multiforme (GBM) being most malignant form, causing 3-4% of all cancer-related deaths (Louis et al. (2007) Acta. Neuropathol. 114: 97-109.). The World Health Organization defines GBM as a grade IV cancer characterized as malignant, mitotically active, and predisposed to necrosis. GBM has a very poor prognosis with a 5-year survival rate of 4-5% with the median survival rate of GBM being 12.6 months (McLendon et al. (2003) Cancer. 98 :1745-1748.). This can be attributed to unique treatment limitations such as a high average age of onset, tumor location, and poor current understandings of the tumor pathophysiology (Louis et al. (2007) Acta. Neuropathol. 114: 97-109). The standard current standard of care for GBM includes tumor resection with concurrent radiotherapy and chemotherapy and in recent years there have been few marked improvements that increase survival rates (Stewart, et al. (2002) Lancet. 359:1011-1018.).

The standard for GBM chemotherapy is temozolomide (TMZ), which is a brain-penetrant alkylating agent that methylates purines (A or G) in DNA and induces apoptosis (Stupp, et al. (2005) N. Engl. J. Med. 352:987-996). However, TMZ use has drawbacks in that significant risk arises from DNA damage in healthy cells and that GBM cells can rapidly develop resistance towards the drug (Carlsson, et al. (2014) EMBO. Mol. Med. 6: 1359-1370).

Epidermal growth factor receptor (EGFR) is a member of the HER superfamily of receptor tyrosine kinases together with ERBB2, ERBB3, and ERBB4. A common driver of GBM progression is EGFR amplification (amp), which is found in nearly 40% of all GBM cases (Hynes et al. (2005) Nat. Rev. Cancer. 5: 341-354; Hatanpaa et al. (2010) Neoplasia. 12:675-684). Additionally, EGFR amplification is associated with the presence of EGFR protein variants: in 68% of EGFR mutants, there is a deletion in the N-terminal ligand-binding region between amino acids 6 and 273. These deletions in the ligand-binding domains of EGFR can lead to ligand-independent activation of EGFR (Yamazaki et al. (1990) Jpn. J. Cancer Res. 81: 773-779.).

Small molecule tyrosine kinase inhibitors (TKIs) are the most clinically advanced of the EGFR-targeted therapies, and both reversible and irreversible inhibitors are in clinical trials. Examples of the reversible inhibitors and irreversible inhibitors include erlotinib, gefitinib, lapatinib, PKI166, canertinib and pelitinib (Mischel et al. (2003) Brain Pathol. 13: 52-61). Mechanistically, these TKIs compete with ATP for binding to the tyrosine kinase domain of EGFR, however, these EGFR-specific tyrosine kinase inhibitors have been relatively ineffective against gliomas, with response rates only reaching as high as 25% in the case of erlotinib (Mischel et al. (2003) Brain Pathol. 13: 52-61; Gan et al. (2009) J. Clin. Neurosci. 16: 748-54). Although TKIs are well tolerated and display some antitumor activity in GBM patients, the recurrent problem of resistance to receptor inhibition limits their efficacy (Learn et al. (2004) Clin. Cancer. Res. 10: 3216-3224; Rich et al. (2004) Nat. Rev. Drug Discov. 3: 430-446). Additionally, recent studies have shown that brain plasma concentrations of gefitinib and erlotinib following therapy were only 6-11% of the starting dose, suggesting that these compounds may be failing to cross the blood-brain barrier as illustrated in table 1 (Karpel-Massler et al. (2009) Mol. Cancer Res. 7 :1000-1012). Thus, insufficient delivery to the target may be another cause of the disappointing clinical results.

Another cause of ineffectiveness of TKIs in treating GBM is that some subjects do not respond to treatment. Timely identifying subjects that do not respond to treatment improves long-term outcomes as treatments can be optimized quickly, thereby reducing the amount of time an ineffective treatment regimen is in place. However, identifying non-responders is particularly complicated in GBM as the presence of a specific genetic alteration in a subject may not be predictive of a therapy's effectiveness in a subject and accessing the tumor repeatedly to determine effectiveness of the therapy is impractical. Previous work demonstrated that aberrant EGFR signaling in GBM leads to upregulated glucose metabolism: a critical metabolic pathway for GBM survival. Given this relationship between a GBM driver oncogene and this essential functional pathway, it is demonstrated herein that molecular imaging, using ¹⁸F-FDG PET, of the dynamics of glucose metabolism with glucose metabolism inhibiting treatment can serve as a rapid, non-invasive predictive biomarker of response in orthotopic, patient-derived GBM xenografts. It is also shown herein that 70% of models do not respond to brain penetrant EFGR TKI JCN068. However, ¹⁸F-FDG PET was able to accurately predict (>90%) a survival response to JCN068 treatment. Importantly, these changes in glucose metabolism were superior at predicting response compared to genotyping EGFR, including polysomy status, in these models. Polysomy generally refers to an alteration from wild type in the copy number of a chromosome or fragment thereof. For example, EGFR polysomy can refer to an increase in EGFR copy number of chromosome 7 (e.g., trisomy 7).

Furthermore, it is demonstrated here that early attenuation of ¹⁸F-FDG uptake with certain EGFR TKIs is associated with reduced tumor progression. Together, these data support that non-invasive measurements of changes in ¹⁸F-FDG uptake can serve as a functional predictive biomarker of response to JCN068 and other EGFR TKIs for the treatment of GBM.

Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics, and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known and those agents whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Administering” or “administration of” a substance, a compound, or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, or transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound, or an agent to a subject will also depend, for example, on the age and/or the physical condition of the subject and the chemical and biological properties of the compound or agent (e.g., solubility, digestibility, bioavailability, stability, and toxicity). In some embodiments, a compound or an agent is administered orally, e.g., to a subject by ingestion. In some embodiments, the orally administered compound or agent is in an extended release or slow release formulation or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer (e.g., a glioblastoma). The skilled worker can readily determine the effective amount for a given situation by routine experimentation.
As used herein, the terms “optional” or “optionally” mean that the subsequently described event or circumstance may occur or may not occur and that the description includes instances where the event or circumstance occurs as well as instances in which it does not. For example, “an optionally substituted alkyl” refers to a molecule or compound in which an alkyl may be substituted as well as where the alkyl is not substituted.
It is understood that substituents and substitution patterns on the compounds disclosed herein can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.
As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂, or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.
As used herein, the term “alkyl” refers to saturated aliphatic groups including, but not limited to, C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl, and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto.

Representative alkoxy groups include methoxy, ethoxy, propoxy, tert-butoxy, and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl,” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y},” when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino” as used herein refers to an amino group substituted with at least one alkyl group.

The term “alkylthio” as used herein refers to a thiol group substituted with an alkyl group and may be represented by the general formula alkylS—.

The term “amide” as used herein refers to a group

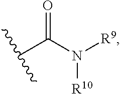

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

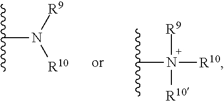

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl” as used herein refers to an alkyl group substituted with an amino group.

The term “aralkyl” as used herein refers to an alkyl group substituted with an aryl group.

The term “aryl” as used herein includes substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

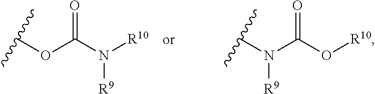

wherein R⁹ and R¹⁰ independently represent hydrogen or a hydrocarbyl group.

The term “carbocyclylalkyl” as used herein refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl” as used herein refers to an alkyl group substituted with a carbocycle group.

The term “carbonate” is art-recognized and refers to a group —OCO₂—.

The term “carboxy” as used herein refers to a group represented by the formula —CO₂H.

The term “ester” as used herein refers to a group —-C(O)OR⁹ wherein R⁹ represents a hydrocarbyl group.

The term “ether” as used herein refers to a hydrocarbyl group linked through an oxygen to another hydrocarbyl group. Accordingly, an ether substituent of a hydrocarbyl group may be hydrocarbyl-O-. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein refer to a halogen group and include chloro, fluoro, bromo, and iodo.

The terms “hetaralkyl” and “heteroaralkyl” as used herein refer to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl,” as used herein, refers to an alkyl group substituted with a heterocycle group.

The terms “heterocyclyl,” “heterocycle,” and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl,” as used herein, refers to a group that is bonded through a carbon atom that does not have a =0 or =S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a=O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl,” as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl,” “polycycle,” and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings.” Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formula:

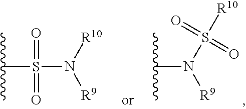

wherein R⁹ and R¹⁰ independently represents hydrogen or hydrocarbyl.

The term “sulfoxide” is art-recognized and refers to the group—S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, and aromatic and non-aromatic substituents of organic compounds. The permissible substituents can be one or more and the same or different for appropriate organic compounds. For purposes of the embodiments described herein, the heteroatoms, such as nitrogen, may have hydrogen substituents and/or any permissible substituents of organic compounds described herein that satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

It will be understood by those skilled in the art that the moieties substituted on the hydrocarbon chain can themselves be substituted, if appropriate.

The term “thioalkyl,” as used herein, refers to an alkyl group substituted with a thiol group.

The term “thioester,” as used herein, refers to a group —C(O)SR⁹ or —SC(O)R⁹, wherein R⁹ represents a hydrocarbyl.

The term “thioether,” as used herein, is equivalent to an ether, wherein the oxygen is replaced with a sulfur.

The term “urea” is art-recognized and may be represented by the general formula

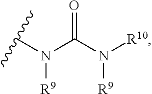

wherein R⁹ and R¹⁰ independently represent hydrogen or a hydrocarbyl.

The term “metabolic process inhibitor” refers to any compound or composition that when administered to a subject, inhibits a metabolic process, either directly (e.g., by inhibiting an enzyme involved in the metabolic process) or indirectly (e.g., by inhibiting the uptake of a substrate to be metabolized). For example, a molecule that inhibits the uptake of glucose into a cell is a glucose metabolism inhibitor.

The term “modulate” as used herein includes the inhibition or suppression of a function or activity (such as cell proliferation) as well as the enhancement of a function or activity. The phrase “pharmaceutically acceptable” is art-recognized. In certain embodiments, the term is used to describe compositions, excipients, adjuvants, polymers, and other materials and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt that is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751; 7,585,851; and 7,964,580, the disclosures of which are incorporated herein by reference. The prodrugs of this disclosure are metabolized to produce a compound of Formula I. The present disclosure includes within its scope prodrugs of the compounds described herein. Conventional procedures for the selection and preparation of suitable prodrugs are described, for example, in “Design of Prodrugs” Ed. H. Bundgaard, Elsevier, 1985.

The terms “Log of solubility,” “LogS,” or “logS” are used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

Types and stages of Gliomas

Primary malignant brain tumors are tumors that start in the brain or spine and are known collectively as gliomas. Gliomas are not a specific type of cancer but rather a term used to describe tumors that originate in glial cells. Examples of primary malignant brain tumors include astrocytomas, pilocytic astrocytomas, pleomorphic xanthoastrocytomas, diffuse astrocytomas, anaplastic astrocytomas, glioblastoma multiformes (GBMs), gangliogliomas, oligodendrogliomas, ependymomas. According to the WHO classification of brain tumors, astrocytomas have been categorized into four grades, determined by the underlying pathology. The characteristics that are used to classify gliomas include mitoses, cellular or nuclear atypia, and vascular proliferation and necrosis with pseudopalisading features. Malignant (or high-grade) gliomas include anaplastic glioma (WHO grade III) as well as glioblastoma multiforme (GBM; WHO grade IV). These are the most aggressive brain tumors with the worst prognosis.

GBMs is the most common, complex, treatment resistant, and deadliest type of brain cancer, accounting for 45% of all brain cancers, with nearly 11,000 men, women, and children diagnosed each year. GBM (also known as grade-4 astrocytoma and glioblastoma multiforme) are the most common types of malignant (cancerous) primary brain tumors. They are extremely aggressive for a number of reasons. First, glioblastoma cells multiply quickly, as they secrete substances that stimulate a rich blood supply. They also have an ability to invade and infiltrate long distances into the normal brain by sending microscopic tendrils of tumor alongside normal cells. Two types of glioblastomas are known. Primary GBM are the most common form; they grow quickly and often cause symptoms early. Secondary glioblastomas are less common, accounting for about 10 percent of all GBMs. They progress from low-grade diffuse astrocytoma or anaplastic astrocytoma and are more often found in younger patients. Secondary GBMs are generally located in the frontal lobe and carry a better prognosis.

GBM is usually treated by combined multi-modal treatment plan including surgical removal of the tumor, radiation, and chemotherapy. First, as much tumor as possible is removed during surgery. The tumor's location in the brain often determines how much of it can be safely removed. After surgery, radiation and chemotherapy slow the growth of remaining tumor cells. The oral chemotherapy drug, temozolomide, is most often used for six weeks, and then monthly thereafter. Another drug, bevacizumab (Avastin®), is also used during treatment. This drug attacks the tumor's ability to recruit blood supply, often slowing or even stopping tumor growth.

Novel investigational treatments are also used and these may involve adding treatments to the standard therapy or replacing one part of the standard therapy with a different treatment that may work better. Some of these treatments include immunotherapy such as vaccine immunotherapies, or low-dose pulses of electricity to the area of the brain where the tumor exists and nano therapies involving spherical nucleic acids (SNAs), such as NU-0129. In some embodiments, the methods of the current disclosure are used in combination with one or more of the aforementioned therapies.

Embodiments of the methods and compositions discussed herein are also contemplated to be applicable to other types of cancers including, but not limited to, lung cancer, non-CNS cancers, CNS cancers, and CNS metastases, such as brain metastases, leptomeningeal metastases, choroidal metastases, spinal cord metastases, and others.

Anticancer Therapies

In some embodiments of the methods described herein, an anti-cancer therapy is one or more of the compounds described in Table 1. Combinations of these compounds or a single compound from Table 1 with other anti-cancer therapies is also contemplated.

TABLE 1
Brain Penetration Rates of the Current Standard of Care Drugs

| | | | | | Brain |
|---|---|---|---|---|---|
| | | | Plasma | CSF | Penetration |
| Compound | Primary | Daily Dose | (ng/ml) | (ng/ml) | rate (%) |
| Afatinib | EGFR- | 50 | 66.7 | 0.46 | 0.7 |
| | mutant | | | | |
| | NSCLC | | | | |
| Alectinib | ALK-mutant | 1200 | 1.5 (unbound | 1.3 | 86.7 |
| | NSCLC | | conc.) | | |
| Crizotinib | ALK-mutant | 500 | 237 | 0.616 | 0.26 |
| | NSCLC | | | | |
| Erlotinib | EGFR- | 150 | 1140 ± 937 | 28.7 ± 16.8 | 2.77 ± 0.45 |
| | mutant | | | | |
| | NSCLC | | | | |
| | EGFR- | 1500 (weekly) | 4445.9 | 51.1 | 1.2 |
| | mutant | | | | |
| | NSCLC | | | | |
| Gefitinib | EGFR- | 250 | 326 ± 116 | 3.7 ± 1.9 | 1.13 ± 0.36 |
| | mutant | | | | |
| | NSCLC | | | | |
| | EGFR- | 750-1000 | 1345.9-5094.4 | 14.7-143.1 | 1.07-3.58 |
| | mutant | | | | |
| | NSCLC | | | | |
| Lapatinib | HER2 + | 1250 | 1515, 3472 | 1.3, 4.5 | 0.09, 0.13 |
| | breast cancer | | | | |

Other brain penetrant TKIs are known in the art, and each of these can be assessed for effectiveness using the methods disclosed herein. For example, International Application No. PCT/US20/22743 discloses compounds of Formula I or Formula I*:

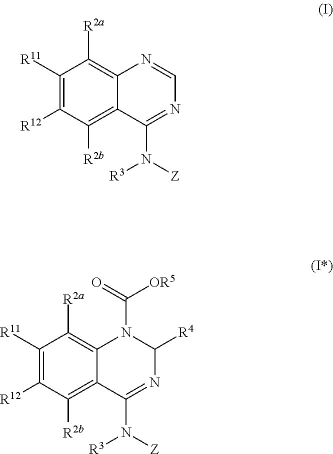

or a pharmaceutically acceptable salt thereof, wherein:

Z is aryl or heteroaryl;

R^{2a} and R^{2b} are each independently selected from hydrogen, alkyl, halo, CN, and NO₂;

R³ is hydrogen, alkyl, or acyl;

R⁴ is alkoxy;

R⁵ is alkyl; R⁷ and R⁸ are, each independently, selected from hydrogen, alkyl, such as alkoxyalkyl, aralkyl, or arylacyl;

R¹¹ is hydrogen, alkyl, halo, CN, NO₂, OR⁷, cycloalkyl, heterocyclyl, aryl, or heteroaryl; and

R¹² is hydrogen, alkyl, halo, CN, NO₂, OR⁸, cycloalkyl, heterocyclyl, aryl, or heteroaryl; or

R¹¹ and R¹² taken together complete a carbocyclic or heterocyclic ring.

In certain preferred embodiments of Formula I or Formula I*, at least one of is R^{2a} and R^{2b} not H. In certain such embodiments of Formula I or Formula I*, if R^{2a} is hydrogen, then R^{2b} is selected from alkyl, halo, CN, and NO₂. In other such embodiments of Formula I or Formula I*, if R^{2b} is hydrogen, then R^{2a} is selected from alkyl, halo, CN, and NO₂.

In certain embodiments of Formula I or Formula I*, the compound is a compound of Formula (IVa) or Formula (IVb):

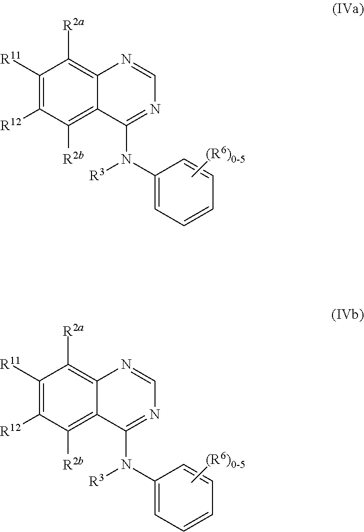

or a pharmaceutically acceptable salt thereof, wherein each instance of R⁶ is independently selected from alkyl, alkoxy, OH, CN, NO₂, halo, alkenyl, alkynyl, aralkyloxy, cycloalkyl, heterocyclyl, aryl, or heteroaryl.

In certain embodiments, of Formula I, I*, Iva, and IVb, R¹¹ is hydrogen. In other preferred embodiments, R¹¹ is OR⁷.

In certain embodiments, of Formula I, I*, Iva, and IVb, R⁷ is hydrogen. In other embodiments, R⁷ is alkyl. In yet other embodiments, R⁷ is alkoxyalkyl. In yet other embodiments, R⁷ is arylacyl.

In certain embodiments, of Formula I, I*, Iva, and IVb, R¹² is heteroaryl, such as furanyl. In certain embodiments, the heteroaryl is substituted with alkyl, alkoxy, OH, CN, NO₂, halo,

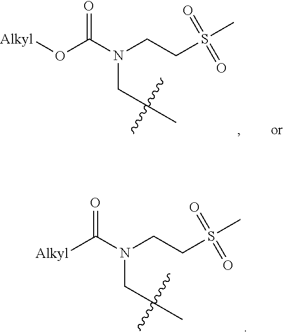

In other preferred embodiments, R¹² is OR⁸.

In certain embodiments of Formula I, I*, Iva, and IVb, R⁸ is hydrogen. In other embodiments, R⁸ is alkyl. In yet other embodiments, R⁸ is alkoxyalkyl. In certain embodiments, R⁸ is alkyl substituted with

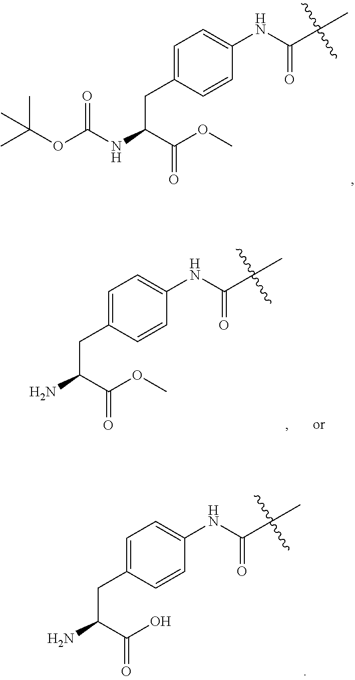 [SMILES: *C(=O)Nc1ccc(C[C@H](NC(=O)OC(C)(C)C)C(=O)OC)cc1 ; *C(=O)Nc1ccc(C[C@H](N)C(=O)OC)cc1 ; *C(=O)Nc1ccc(C[C@H](N)C(=O)O)cc1]

In certain preferred embodiments, of Formula I, I*, Iva, and IVb, R¹¹ and R¹² combine to form a carbocylic or heterocyclic ring, such as a 5-member, 6-member, or 7-member carbocyclic or heterocyclic ring. In certain embodiments, the carbocyclic or heterocyclic ring is substituted with hydroxyl, alkyl (e.g., methyl), or alkenyl (e.g., vinyl).

In certain embodiments, of Formula I, I*, Iva, and IVb, the compound is a compound of Formula Ia, Ib, Ic, or Id:

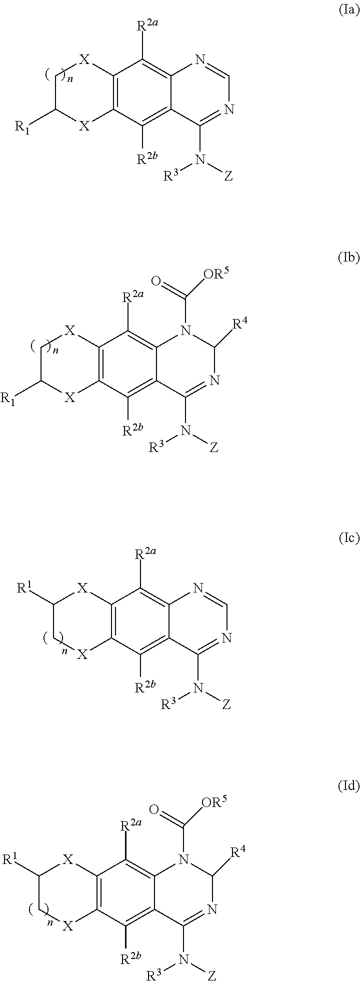

or a pharmaceutically acceptable salt thereof, wherein:

X is O, S, or NH;

Z is aryl or heteroaryl;

R¹ is hydrogen or alkyl;

R^{2a} and R^{2b} are each independently selected from hydrogen, alkyl, halo, CN, and NO₂;

R³ is hydrogen, alkyl, or acyl;

R⁴ is alkoxy;

R⁵ is alkyl; and

n is 0-3.

In certain embodiments of Formula Ia, Ib, Ic, or Id, either R^{2a} or R^{2b} is selected from alkyl, halo, CN, and NO₂. In certain preferred embodiments of Formula Ia, Ib, Ic, or Id, Z is phenyl. In certain preferred embodiments of Formula Ia, Ib, Ic, or Id, X is O. In certain preferred embodiments of Formula Ia, Ib, Ic, or Id, n is 1.

In certain embodiments of Formula Ia, Ib, Ic, or Id, the compound is a compound of Formula (IIa) or Formula (IIb):

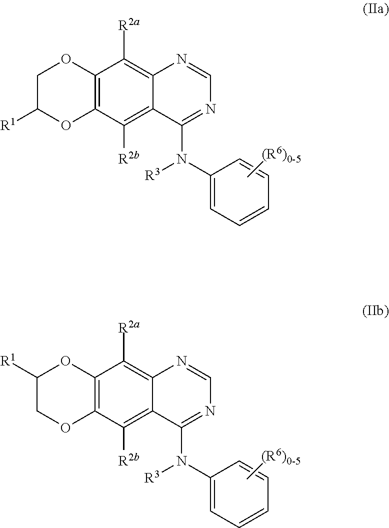

or a pharmaceutically acceptable salt, wherein each instance of R⁶ is independently selected from alkyl, alkoxy, OH, CN, NO₂, halo, alkenyl, alkynyl, aralkyloxy, cycloalkyl, heterocyclyl, aryl, or heteroaryl.

In certain embodiments, wherein R¹ is represented by Formula A:

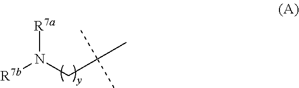

wherein, R^{7a} and R^{7b} are each independently selected from alkyl, alkenyl, alkynl, cycloalkyl, heterocyclyl, aryl or heteroaryl; or R^{7a} and R^{7b} combine to form a heterocyclyl; and y is 0-3.

In certain embodiments of Formula IIa or IIb, R¹ is alkyl (e.g., methyl or ethyl). In certain embodiments, R¹ is substituted with heterocyclyl (e.g., morpholinyl, piperidinyl, pyrrolodinyl, or piperazinyl, such as N-methyl piperazinyl). In other embodiments, R¹ is substituted with amino (e.g., dimethyl amino) In some embodiments, R¹ is alkyl substituted with hydroxyl. In certain preferred embodiments, R¹ is in the S configuration. In other embodiments, R¹ is in the R configuration.

In certain preferred embodiments of Formula IIa or IIb, R³ is hydrogen. In other embodiments, R³ is acyl. In certain embodiments, R³ is alkylacyl. In certain embodiments, R³ is alkyloxyacyl. In certain embodiments, R³ is acyloxyalkyl. In certain embodiments, R³ is

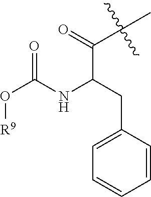

and R⁹ is alkyl.

In certain embodiments of Formula IIa or IIb, Z is aryl or heteroaryl optionally substituted with one or more R⁶; and each instance of R⁶ is independently selected from alkyl, alkoxy, OH, CN, NO₂, halo, alkenyl, alkynyl, aralkyloxy, cycloalkyl, heterocyclyl, aryl, or heteroaryl. In certain preferred embodiments, Z is phenyl substituted with 1, 2, 3, 4, or 5 R⁶. In certain embodiments, each R⁶ is independently selected from halo, alkyl, alkynyl, or arylalkoxy. In even more preferred embodiments, Z is 2-fluoro-3-chlorophenyl, 2-fluorophenyl, 2,3-difluorophenyl, 2,4-difluorophenyl, 2,5-difluorophenyl, 2,6-difluorophenyl, 2,4,6-trifluorophenyl, pentafluorophenyl, 2-fluoro-3-bromophenyl, 2-fluoro-3-ethynylphenyl, and 2-fluoro-3-(trifluoromethyl)phenyl. In other even more preferred embodiments, Z is 3-ethynylphenyl. In yet other even more preferred embodiments, Z is 3-chloro-4-((3-fluorobenzyl)oxy)benzene. In yet other even more preferred embodiments, Z is 3-chloro-2-(trifluoromethyl)phenyl. In yet other even more preferred embodiments, Z is 3-bromophenyl. In yet other even more preferred embodiments, Z is 2-fluoro-5-bromophenyl. In yet other even more preferred embodiments, Z is 2,6-difluoro-5-bromophenyl. In certain embodiments,

Z is substituted with one R⁶ selected from

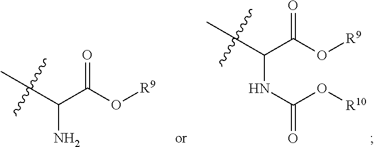

and R⁹ and R¹⁰ are independently selected from alkyl.

In certain embodiments of Formula IIIa or IIIb, the compound is a compound of Formula (IIIa):

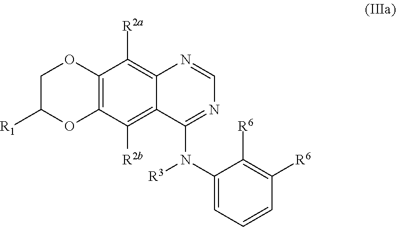

and each R⁶ is independently selected from fluoro, chloro, or bromo.

In certain embodiments of Formula IIIa or IIIb, the compound is a compound of Formula (IIIb):

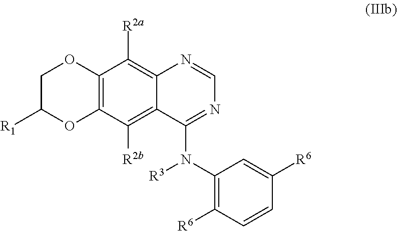

and each R⁶ is independently selected from fluoro, chloro, or bromo.

In certain embodiments of Formula IIIa or IIIb, the compound is a compound of Formula (IIIc):

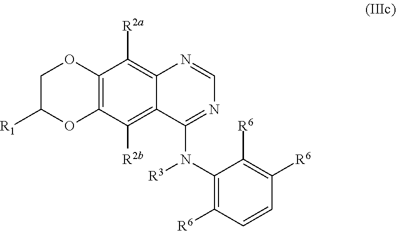

and each R⁶ is independently selected from fluoro, chloro, or bromo.

In certain embodiments of Formula Ia, Ib, Ic, Id, IIa, IIb, IIIc, IIIb, or IIIc, R^{2a} is halo (e.g., fluoro). In some preferred embodiments, R^{2a} is hydrogen.

In certain embodiments of Formula Ia, Ib, Ic, Id, IIa, IIb, IIIc, IIIb, or IIIc, R^{2b} is halo (e.g., fluoro). In other preferred embodiments, R^{2b} is hydrogen.

In certain embodiments of Formula Ia, Ib, Ic, Id, IIa, IIb, IIIc, IIIb, or IIIc, the compound is

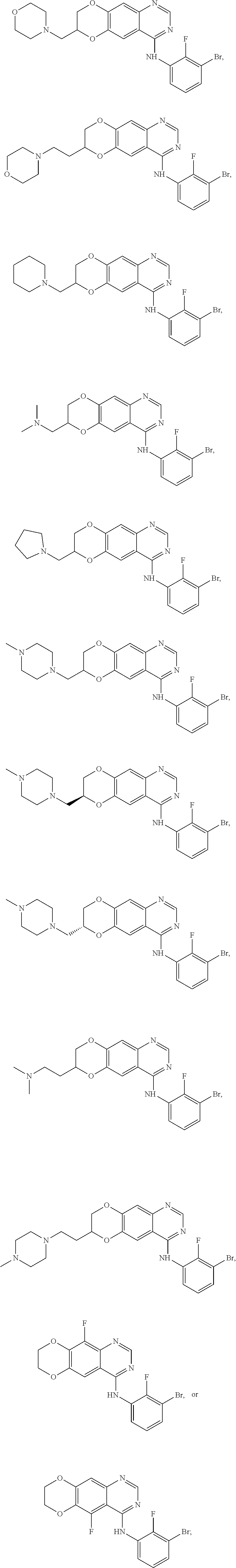

or a pharmaceutically acceptable salt thereof.

In certain embodiments of Formula Ia, Ib, Ic, Id, IIa, IIb, IIIc, IIIb, or IIIc, the compound is

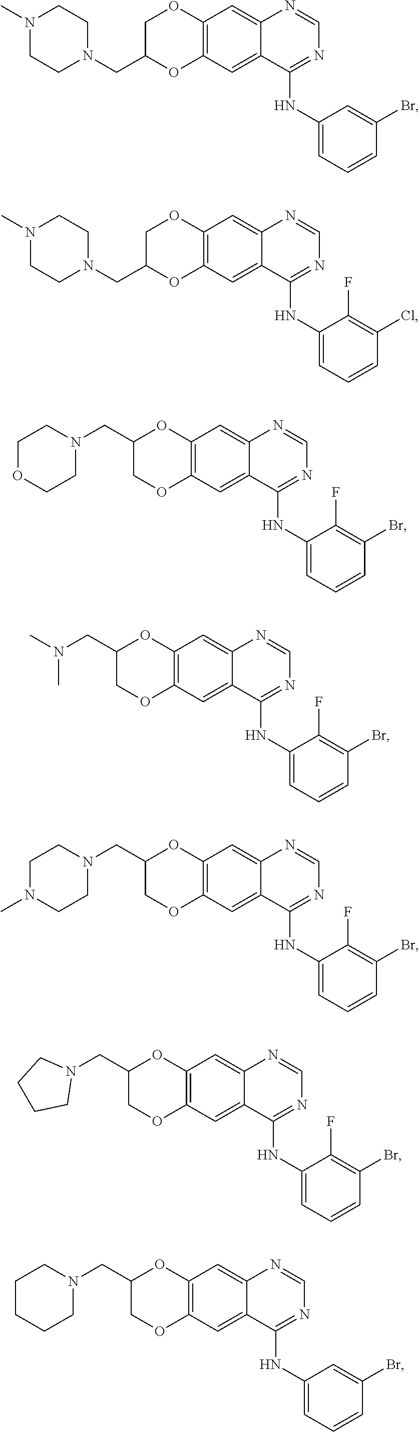

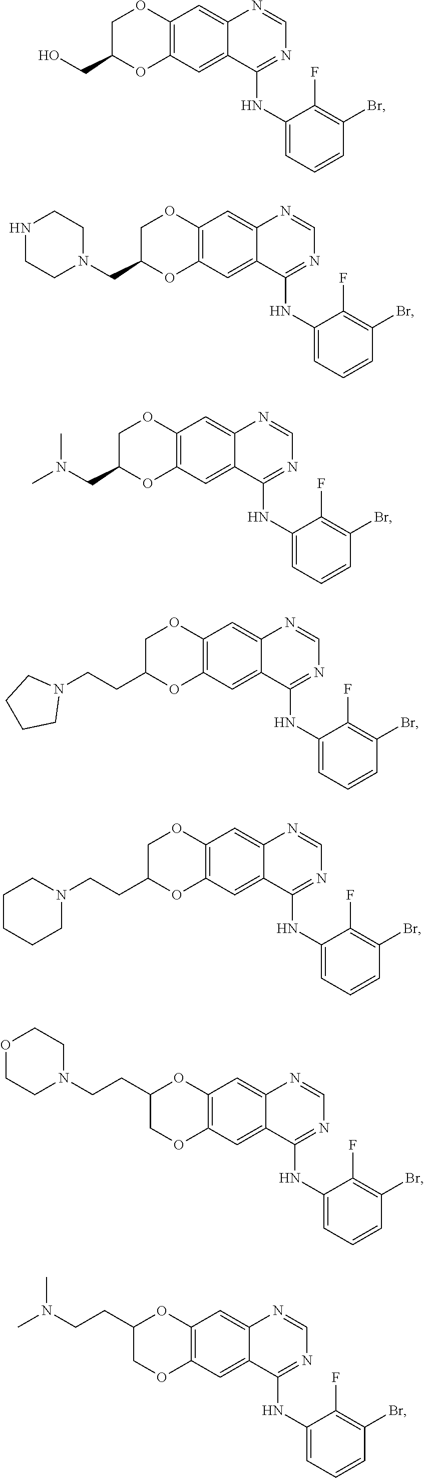

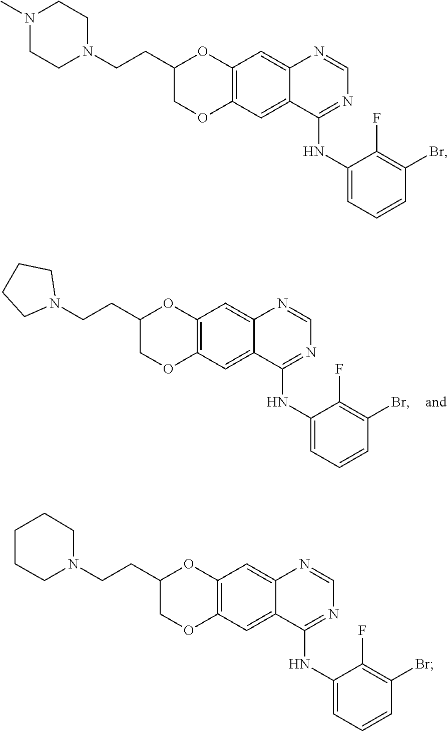 [SMILES: CN1CCN(CCC2COc3cc4c(Nc5cccc(Br)c5F)ncnc4cc3O2)CC1 ; Fc1c(Br)cccc1Nc1ncnc2cc3c(cc12)OCC(CCN1CCCC1)O3 ; "and" ; Fc1c(Br)cccc1Nc1ncnc2cc3c(cc12)OCC(CCN1CCCCC1)O3]

pharmaceutically acceptable salt thereof.

In certain embodiments of Formula I, the compound is

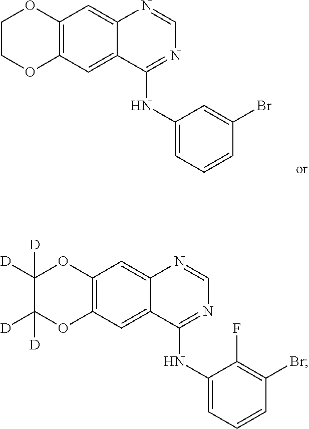 [SMILES: Brc1cccc(Nc2ncnc3cc4c(cc23)OCCO4)c1 ; [2H]C1([2H])Oc2cc3ncnc(Nc4cccc(Br)c4F)c3cc2OC1([2H])[2H]]

or a pharmaceutically acceptable salt thereof.

In certain embodiments of Formula Ia, Ib, Ic, Id, IIa, IIIb, IIIc, IIIb, or IIIc, the compound is

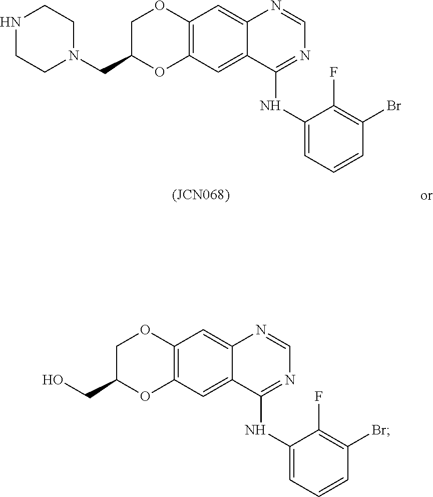 [SMILES: Fc1c(Br)cccc1Nc1ncnc2cc3c(cc12)O[C@@H](CN1CCNCC1)CO3 ; OC[C@H]1COc2cc3ncnc(Nc4cccc(Br)c4F)c3cc2O1]

or a pharmaceutically acceptable salt thereof.

Metabolic Responders

Cancer cells often exhibit changes in metabolic profiles, and these profiles can be analyzed to determine if a cancer cell, tumor, and/or subject having a cancer (e.g., GBM) will respond or not respond to a particular treatment. As used herein, “metabolic responder” refers to a cell, tumor, or subject that exhibits a response to an administered substrate. For example, increased glucose metabolism is observed in some glioblastomas, thus when effectively treated, glucose metabolism in GBM cells is decreased, and glucose uptake by the cells is also decreased. Administering a labeled substrate of glycolysis (e.g., ¹⁸F-fluorodeoxyglucose (¹⁸F-FDG)) allows detection of glucose uptake in a cell, tumor, or subject. Cells, tumors, or subjects that exhibit decreased or eliminated glucose uptake are considered metabolic responders. Conversely, those cells, tumors, or subjects that do not exhibit decreased glucose uptake are considered “metabolic non-responders.” “Responders” can be used interchangeably with “metabolic responders,” and “non-responders” can be used interchangeably with “metabolic non-responders.”

As disclosed herein, metabolic responders to a therapy have improved long-term clinical outcomes. The identification of a metabolic responder can be accomplished shortly after administration of the anti-cancer therapy (e.g., an EGFR TKI). In some embodiments, detection occurs about 1 hour, about 4 hours, about 8 hours, about 12 hours, about 24 hours, about 36, hours, about 48 hours, about 60 hours, or about 72 hours or more after the anti-cancer therapy administration.

Methods of Treatment
The compositions and methods described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In one aspect of the present disclosure, a method is provided for identifying an effective treatment for a subject having a glioblastoma, the method comprising administering to a subject an epidermal growth factor receptor tyrosine kinase inhibitor (EGFR TKI); administering to the subject a substrate for a metabolic process that occurs in the cells of the glioblastoma, wherein the substrate is detectably labeled; and detecting the presence or absence of the detectably labeled substrate in the glioblastoma, wherein a decrease in the detectably labeled substrate relative to a reference level indicates that the glioblastoma is a metabolic responder to the epidermal growth factor receptor tyrosine kinase inhibitor (EGFR TKI), thereby identifying the glucose metabolism inhibitor as an effective treatment. Another aspect of the present disclosure provides a method of treating a glioblastoma comprising administering to a subject a first dose of a metabolic process inhibitor; administering to the subject a detectably labeled substrate for a metabolic process in the cells of the glioblastoma; detecting the presence or absence of the detectably labeled substrate, wherein a decrease in the detectably labeled substrate relative to a reference level indicates that the glioblastoma is a metabolic responder to the metabolic process inhibitor, and administering a second dose of the metabolic process inhibitor.

In certain aspects, the present disclosure provides methods of inhibiting EGFR or AEGFR, comprising administering to a subject an amount of a compound of Table 1 or another compound disclosed herein.

In certain aspects, the present disclosure provides methods of treating cancer comprising administering to a subject in need thereof a compound of Table 1 or another compound disclosed herein and administering a detectably labeled substrate for a metabolic process in the cells of the glioblastoma; detecting the presence or absence of the detectably labeled substrate in the glioblastoma, wherein a decrease in the detectably labeled substrate relative to a reference level indicates that the glioblastoma is a metabolic responder to the administered compound, and administering a second dose of the glucose metabolism inhibitor. In some embodiments, the administered compound is an EGFR-TKI such as erlotinib, gefitinib, icotinib, afatinib, osimertinib, or JCNO68 or a pharmaceutically acceptable salt thereof. In some embodiments, the reference level is a known level. In some embodiments, the glioblastoma comprises a wild type or mutant EGFR or an EGFR with an altered copy number (e.g., trisomy 7). Altered copy number includes polysomy but also alterations in the copy number of a gene that is not due to polysomy (e.g., gene amplification).

In certain aspects, the present disclosure provides methods of treating cancer in a subject, the method comprising administering to the subject a metabolic process inhibitor, an additional agent, and a detectably labeled substrate of a metabolic response, and detecting the label inside the cancer cell or tumor, wherein the metabolic process inhibitor is a compound of the disclosure or a pharmaceutically acceptable salt thereof, and the additional agent is a cytoplasmic p53 stabilizer. In certain embodiments, the cancer is glioblastoma, such as glioblastoma multiforme. In certain embodiments, the method reduces cancer cell proliferation. In certain embodiments, the cancer is relapsed or refractory, while in other embodiments, the cancer is treatment naïve. In certain embodiments, the additional agent is administered, e.g., conjointly with the metabolic process inhibitor, after the subject is identified to be responsive to the metabolic process inhibitor.

In some embodiments of the above aspects, the detectably labeled substrate is administered prior to administration of the anti-cancer therapy. This allows a baseline measurement of uptake of the substrate into the cell to be made, which can then be compared with measurements taken after treatment is administered. For example, in cases in which an EGFR-TKI that inhibits glucose uptake is administered, as glucose uptake decreases in GBM cells, the signal emitted by ¹⁸F-FDG in the glioblastoma also decreases. In some embodiments, failure to detect a decrease in the substrate's label in the cell is indicative of an inadequate response to treatment (i.e., the metabolic process in the cancer cell is not sufficiently affected to adversely impact the cancer cell). Such a cancer cell would be a non-responder to the anti-cancer therapy. If a decrease in the uptake of the substrate is observed, the cancer cell would be a metabolic responder. The determination that a cancer cell or tumor is a metabolic responder indicates that the treatment of the cancer cell or tumor with the anti-cancer therapy is more likely to be effective than the same treatment applied to a non-responder.

In certain embodiments, a subject selected to receive a glucose metabolism inhibitor as an anti-cancer therapy has been determined to be susceptible to the glucose metabolism inhibitor by a method comprising:

a. obtaining a first blood sample from the subject;

b. placing the subject on a ketogenic diet;

c. obtaining a second blood sample from the subject after being placed on a ketogenic diet for a period of time;

d. measuring glucose level in the first and in the second blood sample;

e. comparing the glucose level in the second blood sample with the glucose level in the first blood sample; and

f. determining that the subject is susceptible if the glucose level in the second blood sample is reduced as compared to glucose levels in the first blood sample.

In certain embodiments, the reduction in the glucose level between the second blood sample and the control blood sample is about or greater than 0.15 mM. In certain embodiments, the reduction in the glucose level between the second blood sample and the control blood sample is about or greater than 0.20 mM. In certain embodiments, the reduction in the glucose level between the second blood sample and the control blood sample is in the range of about 0.15 mM to about 2.0 mM. In certain embodiments, the reduction in the glucose level between the second blood sample and the control blood sample is in the range of about 0.25 mM to about 1.0 mM.

In certain embodiments, the cytoplasmic p53 stabilizer is an MDM2 inhibitor. In certain embodiments, the MDM2 inhibitor is a nutlin. In certain embodiments, the MDM2 inhibitor is nutlin-3 or idasanutlin. In certain embodiments, the subject is administered 50 mg to 1600 mg of idasanutlin. In certain embodiments, the subject is administered 100 mg of idasanutlin.

In certain embodiments, the subject is administered 150 mg of idasanutlin. In certain embodiments, the subject is administered 300 mg of idasanutlin. In certain embodiments, the subject is administered 400 mg of idasanutlin. In certain embodiments, the subject is administered 600 mg of idasanutlin. In certain embodiments, the subject is administered 1600 mg of idasanutlin. In other embodiments, the MDM2 inhibitor is RO5045337, RO5503781, RO6839921, SAR405838, DS-3032, DS-3032b, or AMG-232.

In certain embodiments, the cytoplasmic p53 stabilizer is a BCL-2 inhibitor. In certain such embodiments, the BCL-2 inhibitor is antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, or APG-1252.

In certain embodiments, the cytoplasmic p53 stabilizer is a Bcl-xL inhibitor. In certain such embodiments, the Bcl-xL inhibitor is WEHI 539, ABT-263, ABT-199, ABT-737, ABBV-155,sabutoclax, AT101, TW-37, APG-1252, or gambogic acid.

In certain embodiments, the glucose metabolism inhibitor and the cytoplasmic p53 stabilizer are administered in the same composition. In other embodiments, the glucose metabolism inhibitor and the cytoplasmic p53 stabilizer are administered in separate compositions. In certain embodiments, the method further comprises administration of an additional therapy.
Screening Methods
One aspect of the present embodiments relates to screening assays, including xenograft animal model assays. In one embodiment, the assays provide a method for identifying whether a glioblastoma is likely to respond to metabolic process inhibitors disclosed herein and/or combination therapies, such as in a human by using a xenograft animal model assay.
In one embodiment, the present embodiments relate to assays for screening test agents that bind to, or modulate the biological activity of, at least one metabolic process described herein (e.g., labeled substrate of a metabolic substrate). In one embodiment, a method for identifying such an agent entails determining the ability of the agent to modulate, e.g. inhibit, the at least one metabolic described herein (e.g., glycolysis).
Predictive Medicine
The present embodiments also pertain to the field of predictive medicine in which diagnostic assays, prognostic assays, and monitoring clinical trials are used for prognostic (predictive) purposes to thereby treat an individual. Accordingly, one aspect of the present embodiments relates to diagnostic assays for determining the amount and/or activity level of a metabolic process described herein in the context of a glioblastoma to thereby determine whether an individual afflicted with a glioblastoma is likely to respond to inhibitors of one or more metabolic process. Such assays can be used for prognostic or predictive purpose alone, or can be coupled with a therapeutic intervention to thereby prophylactically treat an individual prior to the onset or after recurrence of a glioblastoma. In some aspects the predictive or diagnostic assays can be conducted at the outset of a treatment with an inhibitor, for example, within 1, 2, 3, 4, 5, 6, 7, 8, 9, or 10 days or within 1 week, 2 weeks, 3 weeks or 4 weeks of initially administering the inhibitor. In some aspects, the predictive or diagnostic assays are conducted without assessing impact of the inhibitor on tumor volume, for example. In some instances both predictive and diagnostic assays for metabolism are assessed and tumor volume also is assessed.
Another aspect of the present embodiments pertains to monitoring the influence of agents (e.g., drugs, compounds, and small nucleic acid-based molecules) on the expression or activity of a glioblastoma. For example, imaging of a glioblastoma (e.g., magnetic resonance imaging (MRI)) during a course of treatment to determine changes in tumor volume during the course of treatment. In some embodiments, a first image of the glioblastoma is acquired at or near the time treatment is commenced, and the tumor volume determined from this first image serves as a reference to which later-acquired images can be compared. A reduction in tumor volume during the course of treatment indicate a positive therapeutic response to the selected treatment. The present invention provides for both prophylactic and therapeutic methods of preventing and/or treating glioblastoma that would benefit from a decrease in at least one metabolic process (e.g., glucose metabolism) and an early determination of the effectiveness of such an inhibitor. In some embodiments, which includes both prophylactic and therapeutic methods, the agent is administered in a pharmaceutically acceptable formulation.
Accordingly, one aspect of the present invention provides a method of treating a glioblastoma, the method comprising administering to a subject in need thereof a first dose of an epidermal growth factor receptor tyrosine kinase inhibitor (EGFR TKI), administering to the subject a detectably labeled substrate for a metabolic process, measuring an amount of the detectably labeled substrate in the glioblastoma after administering the EGFR TKI, wherein a decrease in the amount of detectably labeled substrate relative to a reference level indicates that the glioblastoma is a metabolic responder to the inhibitor; and if the glioblastoma is identified as a metabolic responder, treating the subject with the metabolic process inhibitor for a period of time and imaging the glioblastoma to assess a change tumor volume over the period of time, wherein a decrease in tumor volume identifies the inhibitor as an effective treatment for the glioblastoma.
In another aspect, the embodiments pertain to EGFR TKI for use in the treatment of glioblastoma tumor in a subject having a polysomy. In embodiments, the subject responds favorably to the administration of EGFR TKI when administered after the administration of labeled glucose (while labeled glucose is specifically, mentioned any suitable substrate can be utilized herein). In embodiments, the favorable response is determined by the amount of uptake of the labeled glucose in the subject. In further embodiments, the uptake of the labeled glucose is determined by PET (positron emission tomography) imaging. In embodiments, the polysomy is trisomy 7.

In an aspect, the embodiments pertain to labeled glucose for use in determining the efficacy of EGFR TKI in the treatment of glioblastoma tumor, preferably wherein the subject has a polysomy. In embodiments, the polysomy is trisomy 7. In embodiments, the use comprises administering to the subject a first dose of an epidermal growth factor receptor tyrosine kinase inhibitor (EGFR TKI), administering to the subject a detectably labeled glucose for a metabolic process, measuring an amount of the detectably labeled glucose in the glioblastoma after administering the EGFR TKI, wherein a decrease in the amount of detectably labeled glucose relative to a reference level indicates that the glioblastoma is a metabolic responder to the inhibitor; and if the glioblastoma is identified as a metabolic responder, treating the subject with the metabolic process inhibitor for a period of time and imaging the glioblastoma to assess a change tumor volume over the period of time, wherein a decrease in tumor volume identifies the inhibitor as an effective treatment for the glioblastoma.
In another aspect, the embodiments pertain to EGFR TKI for use in the treatment of a glioblastoma tumor in a subject having a polysomy, wherein the amount of labeled glucose uptake determines the efficacy of EGFR TKI treatment in a subject with a polysomy. In embodiments, the polysomy is trisomy 7.
Some aspects relate to uses of a EGFR TKI in the treatment of a cancer subject determined to be a metabolic responder based upon a determination of glucose metabolism. Some embodiments relate to the use of a EGFR TKI and a glucose metabolism substrate to treat a subject that is a metabolic responder based upon a determination of the subject's ability to metabolise the substrate. Some embodiments relate to uses of EGFR inhibitors for, and methods of, treating a glioblastoma in a cancer patient having EGFR polysomy. The methods can include, for example, administering to the patient having a glioblastoma a dose of an epidermal growth factor receptor tyrosine kinase inhibitor (EGFR TKI), where the patient is known to or has been identified as having EGFR polysomy. In some instances, the methods can include the step of identifying or selecting such a patient or determining the polysomy. The methods can include administering to the subject a substrate for a metabolic process, for example a detectably labeled substrate and/or a substrate with an exchangeable proton. The methods further can include measuring or detecting a change or impact on metabolism in the patient. In some instances the measuring can include measuring an amount of the detectably labeled substrate in the glioblastoma after administering the EGFR TKI, wherein a decrease in the amount of detectably labeled substrate relative to a reference level indicates that the glioblastoma is a metabolic responder to the inhibitor. In some instances, if the measured amount is less than the reference level, the methods can include treating the patient with the EGFR TKI for a period of time and optionally imaging the glioblastoma to assess a change in tumor volume over the period of time. In some instances the detecting can include detecting a pH change in the glioblastoma after administering the EGFR TKI, wherein an increase in the pH relative to a reference level indicates that the glioblastoma is a metabolic responder to the inhibitor. Further, if the pH is greater than the reference level, the methods can include treating the subject with the EGFR TKI for a period of time and optionally imaging the glioblastoma to assess a change in tumor volume over the period of time. It should be understood that the EGFR polysomy can be any polysomy, including for example, trisomy 7.
Some embodiments relate to uses of EGFR inhibitors and/or metabolic substrates for, and methods of, identifying or determining whether an EGFR tyrosine kinase inhibitor (EGFR TKI) is a metabolic process inhibitor in a patient with a glioblastoma known or determined to have a polysomy mutation. The methods can include determining or identifying whether the glioblastoma has EGFR polysomy and/or selecting to perform the methods based upon the glioblastoma having EGFR polysomy. The methods can include administering to the patient in need thereof the EGFR TKI based upon knowing or having determined that the patient's glioblastoma comprises EGFR polysomy. In some instances, the methods can include the step of identifying or selecting such a patient or determining the presence of the polysomy prior to administering the EGFR TKI. The methods can include administering to the subject a substrate for a metabolic process, for example a detectably labeled substrate and/or a substrate with an exchangeable proton. The methods further can include measuring or detecting a change or impact on metabolism in the patient. In some instances the measuring can include measuring an amount of the detectably labeled substrate in the glioblastoma after administering the EGFR TKI, wherein a decrease in the amount of detectably labeled substrate relative to a reference level indicates that the glioblastoma is a metabolic responder to the inhibitor. In some instances, if the glioblastoma is identified as a metabolic responder, the methods can include treating the subject with the metabolic process inhibitor for a period of time and optionally imaging the glioblastoma to assess a change tumor volume over the period of time, wherein a decrease in tumor volume identifies the inhibitor as an effective treatment for the glioblastoma. In some instances the detecting can include detecting a pH change in the glioblastoma after administering the EGFR TKI, wherein an increase in the pH relative to a reference level indicates that the glioblastoma is a metabolic responder to the inhibitor. Further, if the pH is greater than the reference level, the methods can include treating the subject with the EGFR TKI for a period of time and optionally imaging the glioblastoma to assess a change in tumor volume over the period of time. It should be understood that the EGFR polysomy can be any polysomy, including for example, trisomy 7.
Some embodiments relate to methods of identifying a subject exhibiting EGFR polysomy as a responder to an EGFR tyrosine kinase inhibitor (EGFR TKI) treatment regimen. The methods can include, for example, selecting or identifying a subject that has cancer, such as glioblastoma or other cancer in the brain, that has EGFR polysomy. The selecting or identifying can include determining whether the subject's cancer has EGFR polysomy. The methods further can include administering the EGFR TKI to the subject and determining whether the EGFR TKI administration results in a change in the metabolism of the cancer where the subject has received a substrate for a metabolic process. The determining whether there is a change can include administering to the subject a substrate for a metabolic process. Any suitable substrate for a metabolic process of the cancer can be used, including for example, a detectably labeled substrate or a substrate with an exchangeable proton. The method can include detecting or measuring a metabolic process in the cancer after administering the EGFR TKI. For example, the metabolic process can include measuring or detecting a change in the presence of the detectably labeled substrate, a change in pH, etc. In the case of the detectably labeled substrate, in some instances, a decrease in the amount of detectably labeled substrate, for example, relative to a reference level, indicates that the subject is a metabolic responder. In some instances in the cases where the substrate comprises an exchangeable proton, an increase in the pH, for example, relative to a reference level, indicates that the subject is a metabolic responder to the inhibitor. In some instances, a subject that is a metabolic responder can be treated with the EGFR TKI for a desired period of time. Optionally, the methods can include imaging the cancer to assess a change in tumor volume over the period of time. It should be understood that the EGFR polysomy can be any polysomy, including for example, trisomy 7.
It should be understood that in some aspects of the above-described embodiments relating to determining a favorable response, determining efficacy of an EGFR TKI treatment and/or in determining efficacy in a subject with a polysomy, the various determining can be done within 1, 2, 3, 4, 5, 6, 7, 8, 9, or 10 days or within 1 week, 2 weeks, 3 weeks or 4 weeks of initially administering the EGFR TKI. In some aspects, the determining can be done, for example, without assessing impact of the EGFR TKI on tumor volume. In some instances the determining can be done and also tumor volume can be assessed.
A decrease in the amount of detectably labeled substrate relative to a reference level between 5% and 100% is sufficient to identify the glioblastoma as a metabolic responder. For example, the decrease can be about 5%, 10%, 20%, 30%, 40%, 50%, 60%, 70%, 80%, 90%, 95%, or even 100%, or any range derivable therein. Imaging of the glioblastoma to determine tumor volume can be done prior to, concurrent with, or after administration of the EGFR TKI or other metabolic process inhibitor. An initial image, at or near the time of administering the inhibitor can provide a tumor volume that serves as a reference point for later determined tumor volume, wherein reduction in tumor volume determined from later images relative to earlier images indicates that the therapy is effective. If the measured amount of the detectably labeled substrate is about the same or greater than the reference level, the treatment may be halted. In some embodiments, wherein the amount of the detectably labeled substrate is about the same or greater than the reference level, a different EGFR TKI or metabolic process inhibitor will be administered to the subject. If treatment with the EGFR TKI or metabolic process inhibitor results in a decreased amount of the detectably labeled substrate in the glioblastoma relative to the reference level, an additional therapy may be administered. In some embodiments, the additional therapy is a cytoplasmic p53 stabilizer, such as an MDM2 inhibitor including, but not limited to nutlin, RO5045337, RO5503781, RO6839921, SAR405838, DS-3032, DS-3032b, or AMG-232. The cytoplasmic p53 stabilizer can be a BCL-2 inhibitor such as, but not limited to antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, or APG-1252. In other embodiments, the cytoplasmic p53 stabilizer is a Bcl-xL inhibitor such as, but not limited to, WEHI 539, ABT-263, ABT-199, ABT-737, ABBV-155, sabutoclax, AT101, TW-37, APG-1252, or gambogic acid. In all embodiments of the present invention wherein a combination of therapies is administered, they can be administered conjointly.
In some embodiments, the EGFR TKI is one or more of the EGFR TKI is erlotinib, gefitinib, icotinib, afatinib, osimertinib, or an EGFR TKI of Formula I or Formula I* disclosed herein, or a pharmaceutically acceptable salt thereof. The methods disclosed herein allow early determination, and subsequent confirmation, of effective therapies for treating glioblastoma. Even with this advancement in treating glioblastomas, other available information can be included in the methods. For example, the methods of treatment may be specifically tailored or modified based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype” or “drug response genotype”). Thus, another aspect encompassed by the present invention provides methods for tailoring a subject's treatment with agents described according to that individual's drug response genotype. Pharmacogenomics allows a clinician or physician to target prophylactic or therapeutic treatments to patients who will most benefit from the treatment and to avoid treatment of patients who will experience toxic drug-related side effects.

Methods of Assessment

Glucose Uptake Tests

In embodiments of the methods and compositions of the current disclosure, the glioblastoma or cancer is classified to be either a “metabolic responder” or a “metabolic non-responder,” i.e., determined to be susceptible to glucose metabolism inhibitors. In certain embodiments, the classification of the cancer is prior to administering to the subject a treatment comprising a glucose metabolism inhibitor and optionally a cytoplasmic p53 stabilizer. Accordingly, the current disclosure provides for methods for assessing and classifying a cancer, determining the susceptibility of a subject to treatments involve analysis of glucose metabolism, glycolysis, or glucose uptake. Techniques to monitor glycolysis and glucose uptake is provided by T. TeSlaa and M. A. Teitell. 2014. Methods in Enzymology, Volume 542, pp. 92-114, incorporated herein by reference.

In some aspects, the classification of a cancer as a metabolic responder or non-responder is after administration to the subject of a treatment glucose metabolism inhibitor. For example, in some embodiments, classification comprises administering to a subject in need thereof a first dose of a glucose metabolism inhibitor; administering to the subject a detectably labeled substrate for a metabolic process in the cells of the glioblastoma; detecting the presence or absence of the detectably labeled substrate in the glioblastoma, wherein a decrease in the detectably labeled substrate relative to a reference level indicates that the glioblastoma is a metabolic responder to the glucose metabolism inhibitor. In some embodiments, the substrate is a monosaccharide, polysaccharide, or a lipid. For example, in some embodiments, the monosaccharide is glucose or fluorodeoxyglucose (¹⁸F-FDG). In such embodiments, the metabolic process that acts on the substrate is glycolysis. In some embodiments, the substrate is a lipid such as, but not limited to, a fatty acid, a triglyceride, or a phospholipid. In some embodiments, the classification comprises detecting the labeled substrate prior to administering the glucose metabolism inhibitor to the subject. In some embodiments, detecting the labeled substrate comprises performing a PET scan. In some embodiments, an additional PET scan is performed prior to administering the glucose metabolism inhibitor to the subject. An initial PET scan can serve as the base line measurement of glucose uptake in a cell. Thus, in some embodiments, the reference level is the level of metabolite detected in the PET scan prior to administering the glucose metabolism inhibitor. As demonstrated herein, results from ¹⁸F-FDG PET indicating that a subject's cancer (e.g., glioblastoma) is a metabolic responder can be a more accurate predictor of survival in response to certain treatments (e.g., TKI therapy) compared to genetic analysis of the cancer (e.g., polysomy status). As noted previously, the determination of whether the cancer is a metabolic responder or not can be done at the outset of a treatment with a cancer drug. For example, the determination can be done within 1, 2, 3, 4, 5, 6, 7, 8, or 9 or 10 days or within 1 week, 2 weeks, 3 weeks or 4 weeks of initially administering the cancer drug. In some aspects, the determining can be conducted without assessing impact of the cancer drug on tumor volume, for example. In some instances the determination of metabolic response to the drug is done and also the tumor volume is assessed.

Glycolysis is the intracellular biochemical conversion of one molecule of glucose into two molecules of pyruvate with the concurrent generation of two molecules of ATP. Pyruvate is a metabolic intermediate with several potential fates including entrance into the tricarboxylic acid (TCA) cycle within mitochondria to produce NADH and FADH2. Alternatively, pyruvate can be converted into lactate in the cytosol by lactate dehydrogenase with concurrent regeneration of NAD from NADH. An increased flux through glycolysis supports the proliferation of cancer cells by providing, for example, additional energy in the form of ATP as well as glucose-derived metabolic intermediates for nucleotide, lipid, and protein biosynthesis. Warburg (Oncologia. 1956;9(2):75-83) first observed that proliferating tumor cells augment aerobic glycolysis, the conversion of glucose to lactate in the presence of oxygen, in contrast to nonmalignant cells that mainly respire when oxygen is available. This mitochondrial bypass, called the Warburg effect, occurs in rapidly proliferating cells including cancer cells, activated lymphocytes, and pluripotent stem cells. The Warburg effect has been exploited for clinical diagnostic tests that use positron emission tomography (PET) scanning to identify increased cellular uptake of fluorinated glucose analogs such as ¹⁸F-fluorodeoxyglucose.

Thus, glycolysis represents a target for therapeutic and diagnostic methods. In the context of the current methods, which can be in vitro or in vivo methods, the measurement of glucose uptake and lactate excretion by malignant cells may be useful to detect shifts in glucose catabolism and/or susceptibility to glucose metabolism inhibitors. Detecting such shifts is important for methods of treating GBM, methods of reducing the risk of ineffective therapy, methods for reducing the chances of tumor survival. For the purposes of this disclosure, ¹⁸F-FDG PET serves in certain embodiments as a rapid non-invasive functional biomarker to predict sensitivity to p53 activation. This non-invasive analysis could be particularly valuable for malignant brain tumors where pharmacokinetic/pharmacodynamics assessment is extremely difficult and impractical. In some cases, delayed imaging protocols (41) and parametric response maps (PRMs) with MRI fusion can be useful for quantifying the changes in tumors ¹⁸F-FDG uptake (42).

In certain aspects, the methods can relate to measuring glucose uptake and lactate production. For cells in culture, glycolytic flux can be quantified by measuring glucose uptake and lactate excretion. Glucose uptake into the cell is through glucose transporters (Glut1-Glut4), whereas lactate excretion is through monocarboxylate transporters (MCT1-MCT4) at the cell membrane.

Extracellular Glucose and Lactate

Methods to detect glucose uptake and lactate excretion include, for example, extracellular glucose or lactate kit, extracellular bioanalyzer, ECAR measurement, [3H]-2-DG or [14C]-2-DG uptake, ¹⁸F-FDG uptake, or 2-NBDG uptake.

Commercially available kits and instruments are available to quantify glucose and lactate levels within cell culture media. Kit detection methods are usually colorimetric or fluorometric and are compatible with standard lab equipment such as spectrophotometers.

BioProfile Analyzers (such as Nova Biomedical) or Biochemistry Analyzers (such as for example YSI Life Sciences) can measure levels of both glucose and lactate in cell culture media. GlucCell (Cesco BioProducts) can measure only glucose levels in cell culture media. While each commercial method has a different detection protocol, the collection of culture media for analysis is the same.

Extracellular Acidification Rate

Glycolysis can also be determined through measurements of the extracellular acidification rate (ECAR) of the surrounding media, which is predominately from the excretion of lactic acid per unit time after its conversion from pyruvate. The Seahorse extracellular flux (XF) analyzer (Seahorse Bioscience) is a tool for measuring glycolysis and oxidative phosphorylation (through oxygen consumption) simultaneously in the same cells.

Glucose Analog Uptake

Certain embodiments of the methods of the current disclosure include the use of glucose analogs. As would be familiar to a person skilled in the art, to determine the glucose uptake rate by cells, a labeled isoform of glucose can be added to the cell culture media and then measured within cells after a given period of time. Exemplary types of glucose analogs for these studies include but are not limited to radioactive glucose analogs, such as 2-deoxy-D-[1,2-3H]-glucose, 2-deoxy-D-[1-14C]-glucose, or 2-deoxy-2-(¹⁸F)-fluoro-D-glucose (¹⁸FDG), or fluorescent glucose analogs, such as 2-[N-(7-nitrobenz-2-oxa-1,3-diaxol-4-yl)amino]-2-deoxyglucose (2-NBDG). Measurements of radioactive glucose analog uptake require a scintillation counter, whereas 2-NBDG uptake is usually measured by flow cytometry or fluorescent microscopy. In some embodiments, the glucose uptake is measured by the uptake of radio-labelled glucose 2-deoxy-2-[fluorine-18]fluoro-D-glucose (¹⁸F-FDG). In further embodiments, detecting the ¹⁸F-FDG is by positron emission tomography (PET). In some embodiments, the biopsy is taken from a GBM tumor. A detailed description of an example of measuring ¹⁸F-FDG is provided in the examples below.

Amino-acid Analog Uptake

Certain embodiments, of the methods of the current disclosure include the use of amino acid analogs. As would be familiar to a person skilled in the art, to determine the amino acid uptake rate by cells, a labeled isoform of an amino acid (e.g., L-DOPA) can be added to the cell culture media and then measured within cells after a given period of time. Exemplary types of glucose analogs for these studies include but are not limited to radioactive glucose analogs, such as 3,4-dihydroxy-6-[¹⁸F]-fluoro-L-phenylalanine (¹⁸F-FDOPA). In some embodiments, amino acid uptake is measured by the uptake of radio-labelled amino acids (e.g., ¹⁸F-DOPA, -¹⁸F-Fluoro-Ethyl-Tyrosine (¹⁸F-FET)). In further embodiments, detecting the radiolabeled amino acid is by positron emission tomography (PET).

### CEST-MRI

In certain aspects, changes in pH in a cancer are assessed to determine if that cancer is a metabolic responder to treatment. Chemical exchange saturation transfer-magnetic resonance imaging (CEST-MRI) allows in vivo detection of pH changes that result when magnetization is transferred from a targeted species to water molecules. This transfer requires that the targeted species have a ¹H proton that it can exchange with water. CEST-MRI can be used to detect pH changes in the tumor environment, thus allowing non-invasive determination if a subject's cancer is a metabolic responder to treatment. Certain metabolites (e.g., glutamate, gamma-aminobutyric acid (GABA), glycine, and myo-inositol (MI)) have exchangeable protons that have distinct chemical shifts, which allow sensitive detection and pH measurement.

In certain aspects, the methods can relate to comparing analyte (e.g., glucose or L-DOPA) uptake by a biological sample such as a tumor sample with a control. Fold increases or decreases may be, be at least, or be at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 20 55%, 60%, 65%, 70%, 75%, 80%, 85%, 90%, 95%, 100% or more, or any range derivable therein. Alternatively, differences in expression between a sample and a reference may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein. Other ways to express relative expression levels are with normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 30 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein. In some embodiments, the levels can be relative to a control.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered in some embodiments.

Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins, or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste. To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), or a surface-active or dispersing agent. Molded tablets may be made by molding in a suitable machine a mixture of the powdered compound moistened with an inert liquid diluent. The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders, which may be reconstituted into sterile injectable solutions or dispersions just prior to use, which may contain antioxidants, buffers, bacteriostats, solutes that render the formulation isotonic with the blood of the intended recipient, or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier. Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt, or amide thereof, the route of administration, the time of administration, the rate of excretion of the particular compound(s) being employed, the duration of the treatment, other drugs, compounds, and/or materials used in combination with the particular compound(s) employed, the age, sex, weight, condition, general health, and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent. Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions. Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

The invention now being generally described, it will be more readily understood by reference to the following examples which are included merely for purposes of illustration of certain aspects and embodiments of the present invention, and are not intended to limit the invention.

Predictive Value of EGFR Genetic Alterations on Response to Therapy

Multiple variants of EGFR have been associated with GBM. For example, EGFRvIII, also known as de2-7EGFR and AEGFR, comprises an in-frame deletion of 801 base pairs in exons 2-7 that removes 267 amino acids from the extracellular domain. This results in creation of a junction site between exons 1 and 8 and a new glycine residue. Copy number alterations (CAN) of EGFR, i.e., polysomy and amplification, variants have also been observed in GBM.

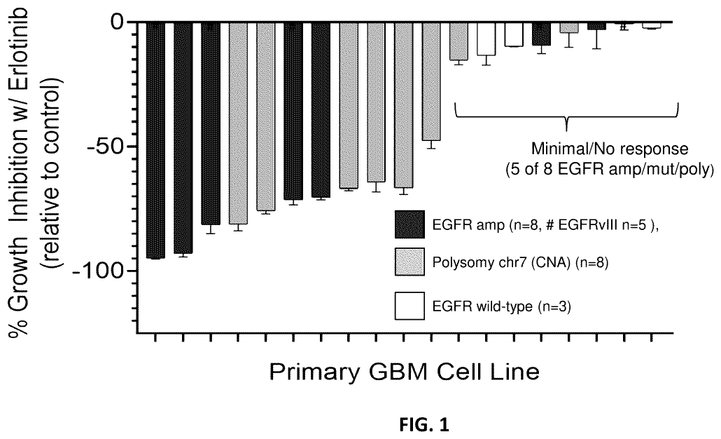

Changes in glucose consumption with acute EGFR inhibition across 19 patient-derived GBM cell lines with either wild type, EGFRvIII, or copy number alterations was characterized. The cells were cultured in supplemented serum-free medium as gliomaspheres which, in contrast to serum-based culture conditions, preserve many of the molecular features of patient tumors. Treatment with the EGFR tyrosine kinase inhibitor (EGFRi) erlotinib identified a subset of GBMs whose radio-labeled glucose uptake (¹⁸F-FDG) was significantly attenuated with EGFR inhibition; hereafter termed “metabolic responders” Of the 19 cell lines, 8 showed minimal to no response to erlotinib (FIG. 1). Of the 8 cell lines that were non-responders to erlotinib, 5 had tumors with variant EGFR and 3 had wild type EGFR. Taken together, these data illustrate two key points. First, acute inhibition of EGFR rapidly attenuates glucose utilization in a subset of primary GBM cells, and second, genetic alterations in EGFR could not alone predict which GBMs have a metabolic response to EGFRi.

Reduction of Glucose Uptake is Observed in GBM Cells Responsive to Therapy

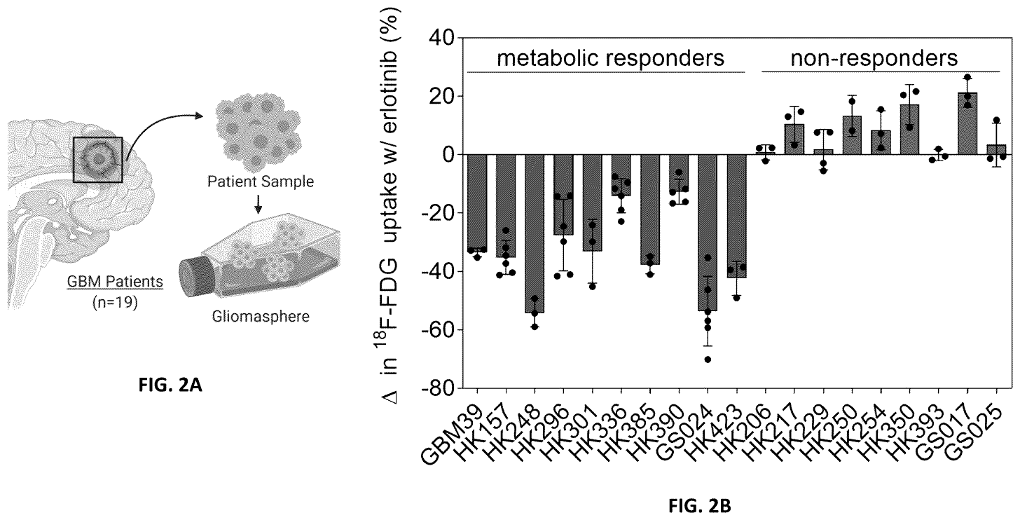

Glucose uptake has been previously shown to be increased in GBM cells. To determine if glucose uptake is correlated with a response to therapy, GBM cells were obtained by biopsy from patients and cultured to generate gliomaspheres. Biopsy samples were minced using a no. 10 scalpel and further dissociated using the MACS Brain Tumor Dissociation Kit (Miltenyi Biotec). Briefly, tumor samples were suspended in the digestion cocktail and dissociated in gentleMACS C tubes with program h_tumor_01.01 followed by h_tumor_02.01. Samples were incubated at 37° C. for 30 minutes followed by another run of h_tumor_02.01. Samples were strained through a 70 μm filter and red blood cells were lysed in ACK buffer. Samples were then incubated with Myelin Removal Beads II (Miltenyi Biotec) and removed using LS columns (Miltenyi Biotec) in a MACS magnetic separater. Next, samples were incubated with CD45+ Removal Beads (Miltenyi Biotec) and removed in the same manner. Purified tumor cells were then cultured in gliomasphere media. Cells were plated at 5×10⁴ cells/mL and treated with erlotinib for the indicated times. After the appropriate treatments, cells were collected and resuspended in glucose-free DMEM/F12 (US Biological) containing ¹⁸F-FDG (radioactivity 1 μCi/mL). Cells were incubated at 37 ° C. for 1 h and then washed three times with ice-cold PBS. The radioactivity of each sample was then measured with a gamma counter. Referring to FIG. 2, clear delineation can be made between samples that responded to treatment and those that did not respond to treatment. Specifically, gliomaspheres were determined to be “metabolic responders” when ¹⁸F-FDG uptake was decreased after treatment, and “non-responders” showed little change or an increase in ¹⁸F-FDG uptake after treatment.

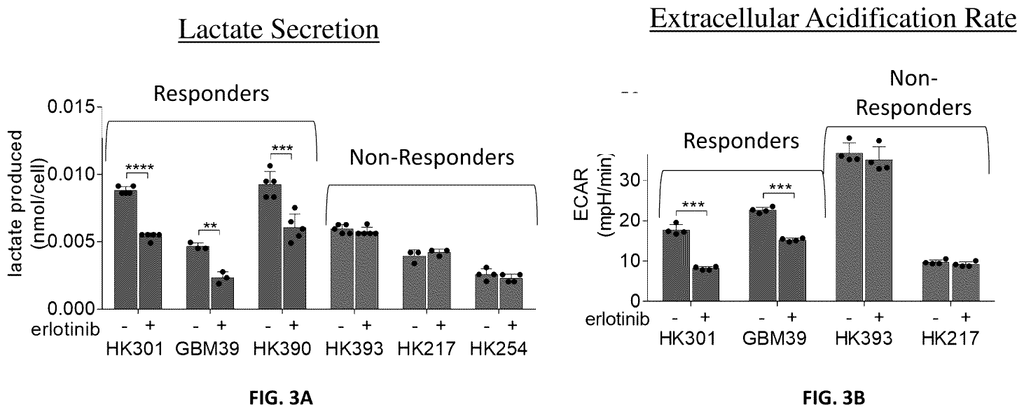

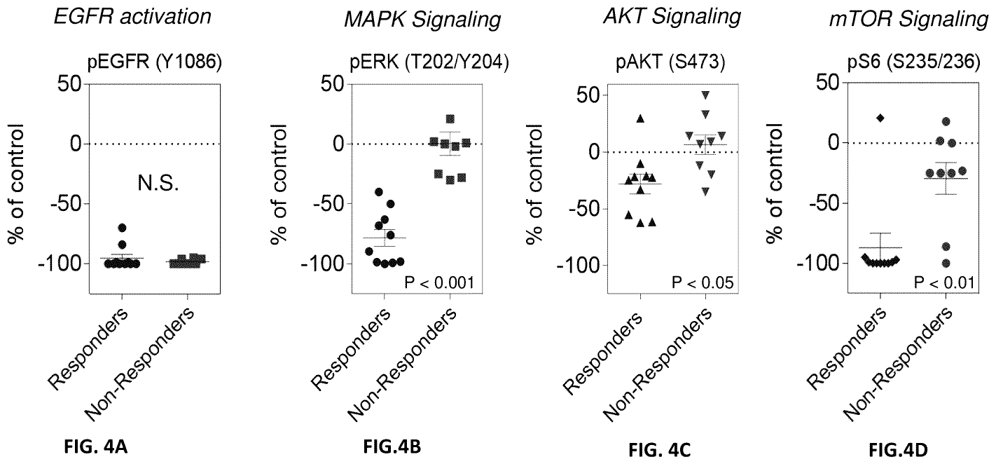

Lactate secretion was measured in a subset of the responders and non-responders as defined by the ¹⁸F-FDG uptake assay (FIG. 3A). Responders secreted less lactate after treatment with erlotinib, confirming that the FGD substrate is a good surrogate for measuring changes in glycolysis. The rate of extracellular acidification (ECAR) was also measured in the cells by chemical exchange saturation transfer (CEST)-MRI. As shown in FIG. 3B, responder cells treated with erlotinib showed decreased ECAR relative to untreated cells. Non-responders showed no change in ECAR after treatment with erlotinib.

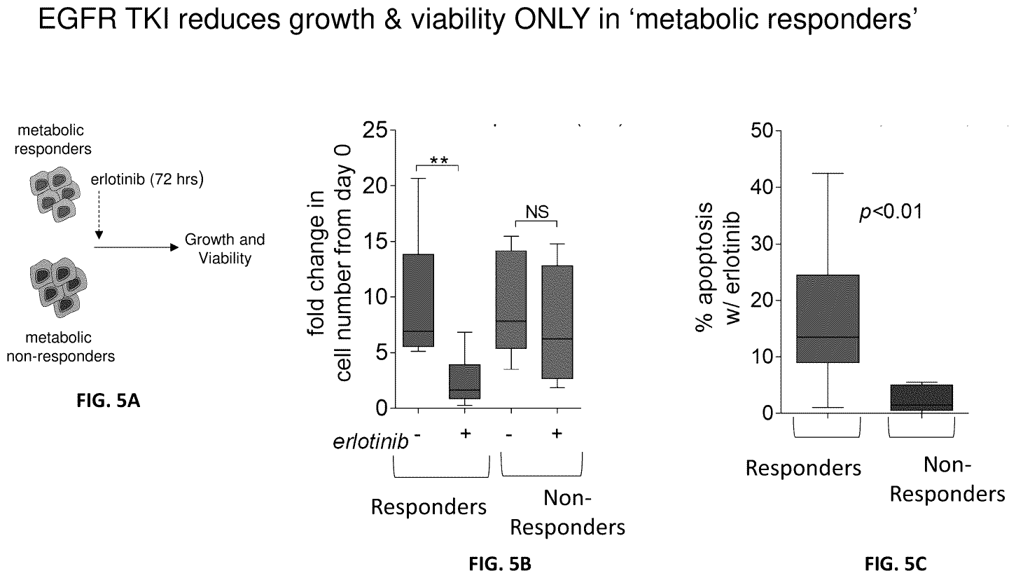

The gliomaspheres were assayed 72 hours after erlotinib treatment to assess changes in growth and viability (FIG. 5A). Perturbations in glucose metabolism can induce the expression of pro-apoptotic factors and stimulate intrinsic apoptosis, suggesting that reduced glucose uptake in response to EGFRi would stimulate the intrinsic apoptotic pathway. A significant reduction in the fold change of the number of cells in the gliomaspheres was observed in the responders, whereas no significant change was observed in the non-responders (FIG. 5B). Apoptosis was significantly increased in the metabolic responders relative to the non-responders (FIG. 5C). Thus, EGFR TKI therapy reduced the growth and viability of GBM cells in metabolic responders, but not in non-responders.

The cultured gliomaspheres were assessed to determine if there was a correlation between genetic variation in the gliomaspheres and metabolic response. DNA was harvested from the gliomaspheres and sequenced to detect variation in CDKN2A, EGFR, MGM2, MDM4, PTEN, NF1, RB 1, and p53. The library construction was performed with the SeqCap EZ System from NimbleGen according to the manufacturer's instructions. Briefly, genomic DNA was sheared and size-selected to approximately 300 bp, and the ends were repaired and ligated to specific adapters and multiplexing indexes. Fragments were then incubated with SeqCap biotinylated DNA baits after LM-PCR, and the hybrids were purified with streptavidin-coated magnetic beads. After amplification of 18 or fewer PCR cycles, the libraries were then sequenced on the HiSeq 3000 platform from Illumina, using 150-bp paired-end reads.

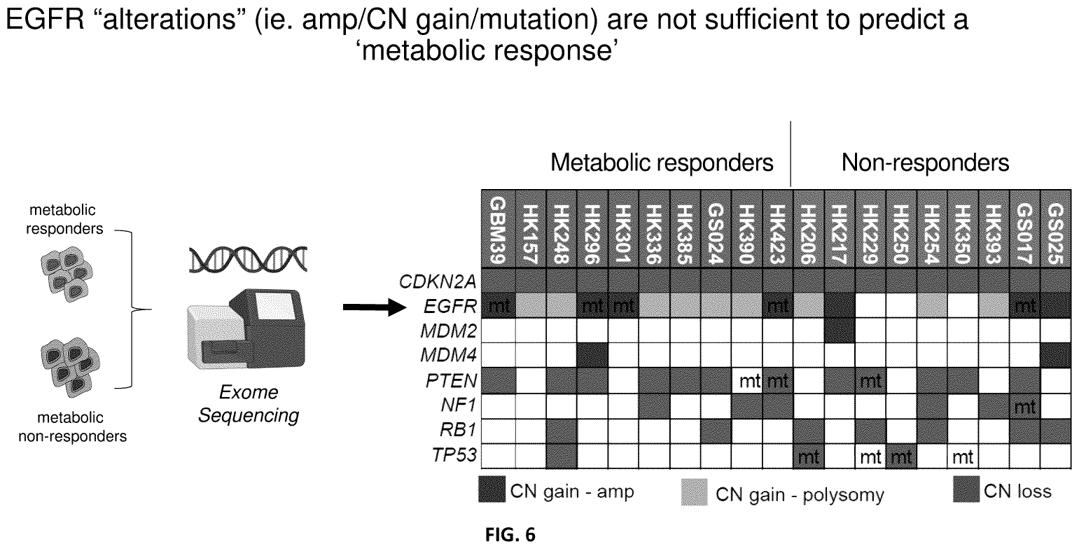

The sequence data were aligned to the GRCh37 human reference genome with BWA v0.7.7-r411. PCR duplicates were marked with the MarkDuplicates program in the Picard-tools-1.115 tool set. GATK v3.2-2 was used for insertions and deletions (INDEL) realignment and base quality recalibration. Exome coverage was calculated with bedtools. Samtools was used to call the single-nucleotide variants (SNVs) and small INDELs. Varscan2 was used to call the somatic SNVs. All variants were annotated with the Annovar program. Referring to FIG. 6, the observed genetic variation in these genes was insufficient to predict metabolic response. Regarding EGFR, although amplification and polysomy were more prevalent relative to the other genes, this variation was not predictive of a responder status.

¹⁸F-FDG PET Rapidly Predicts Therapeutic Outcome In Vivo

As shown above, genetic variation is insufficient to predict a metabolic response to therapy (FIG. 6) or a response to therapy generally (FIG. 1). Conversely, glucose uptake does identify metabolic responders (FIG. 2). Because treatment was shown to reduce growth and viability only in metabolic responders in vitro (FIGS. 5B, 5C), it was invested whether glucose uptake predicts therapeutic outcome in vivo.

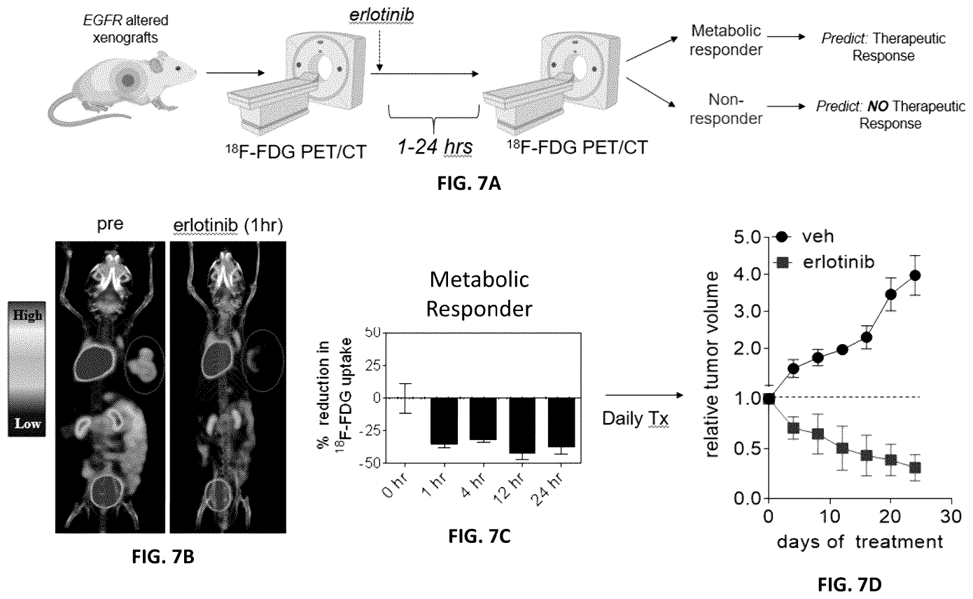

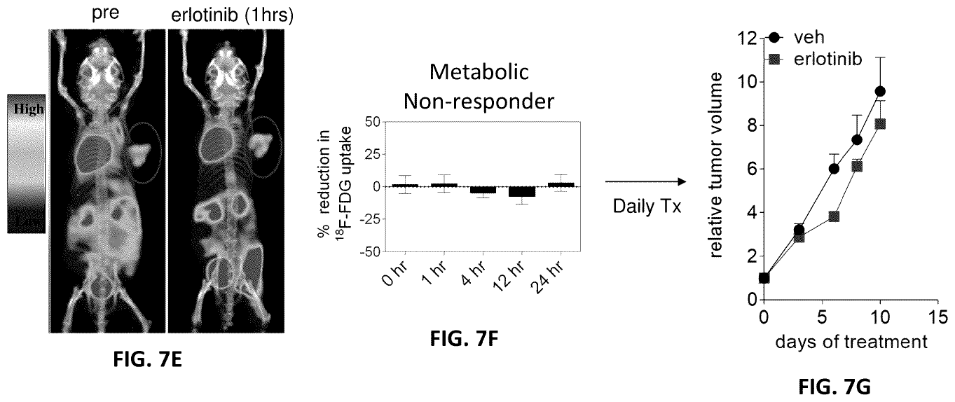

Mice carrying EGFR altered xenografts were assayed for glucose uptake by ¹⁸F-FDG PET scanning. For baseline ¹⁸F-FDG scans, mice were treated with vehicle, anesthetized with 2% isoflurane, and intravenously injected with 70 μCi of ¹⁸F-FDG. Mice were imaged with a G8 PET/CT scanner (Sofie Biosciences). All mice were then dosed with erlotinib (75 mg/kg) and rescanned 1, 4, 12, and 24 hours after treatment (FIG. 7A). Imaging before and after treatment allowed for detection of changes in ¹⁸F-FDG uptake in the mice xenographs. For example, FIG. 7B shows a mouse xenograft (circled) before and 1 hour after erlotinib treatment. A consistent reduction of at least 25% in ¹⁸F-FDG uptake was observed for each time point relative to the pretreatment level (0 hr) of the mouse (FIG. 7C). Thus, after just 1 hour, a determination was made that the xenograft was a metabolic responder.

Growth of metabolic responder xenografts was assayed for approximately 25 days in mice treated with erlotinib or with vehicle. A distinct difference was observed between the treatment groups with the control group exhibiting tumor growth at each time point, whereas the erlotinib treated tumors showed decreased tumor volume at each time point (FIG. 7D).

In comparison, FIG. 7E shows the ¹⁸F-FDG PET scans of a non-responder xenograft. The pre- and post-treatment scans show no distinct change in the xenograft (FIG. 7F). Non-responder mice treated with erlotinib or with vehicle both show the same trend of increasing tumor volume (FIG. 7G), indicating that the metabolic non-responder xenografts were also not responding to treatment. These results indicate that responder status can be determined by ¹⁸F-FDG PET scans very shortly after treatment initiation and that such responder status is predictive of responsiveness to therapy.

JCNO68, like Other EGFR TKIs, Rapidly Inhibits Glucose Metabolism Specifically in GBM Cells and Effectively Treats GBM Cells

Assessing the effectiveness of a GBM therapy in a patient can be exceeding difficult due to the impracticality of accessing the tumor for biopsy or other detect inspection methods. Further, there are no known methods for predicting an in vivo response of a GBM to treatment. For these reasons, the ability of glucose uptake to serve as a predictor of treatment efficacy in vivo was investigated.

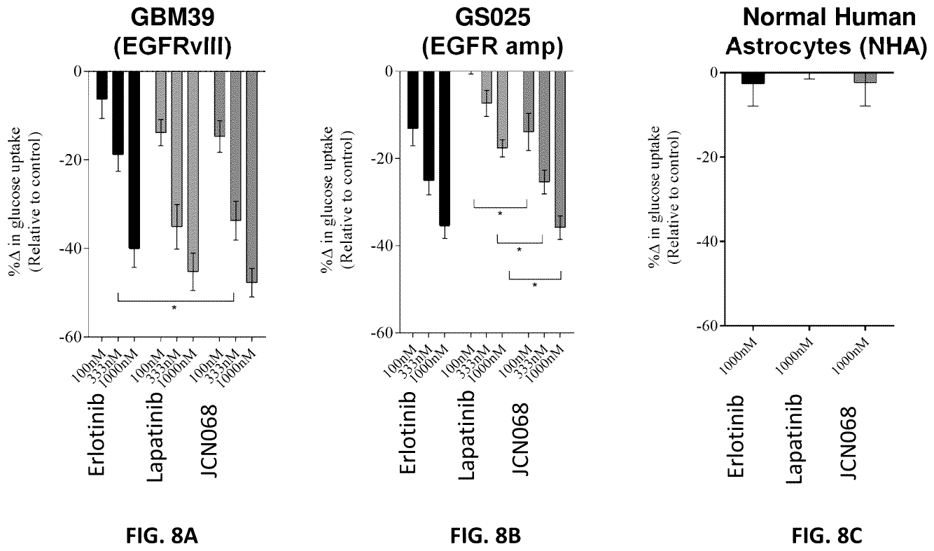

To determine if glucose can identify metabolic responders and non-responders to EGFR TKI treatment, two GBM cell lines were compared to normal human astrocytes. GBM39, which has the common EGFRvIII variant, showed decreasing glucose uptake with increasing concentrations of erlotinib, lapatinib, and JCNO68, a brain penetrant EGFR TKI (FIGS. 8A-8C). This pattern was also observed in GS025, a GBM cell line having an EGFR amplification variant. Conversely, little to no change was observed for any of these drugs in normal human astrocytes. This illustrates that JCNO68 inhibits glucose metabolism in GBM cells, but not in normal astrocytes.

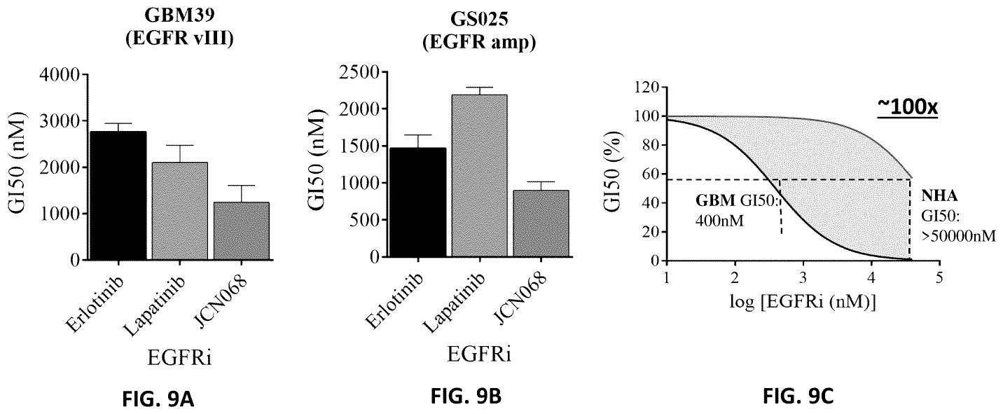

These rapid changes in glucose metabolism were observed with an effective response to treatment as measured by inhibition of cellular proliferation. Of the three drugs tested, JCNO68 had the lowest concentration necessary to achieve 50% of maximal inhibition of cell proliferation (GI50), regardless of cell type (FIGS. 9A and 9B). Further, the GI50 for the treated GBM cells was over 100-fold lower than that observed for the normal human astrocytes (FIG. 9C).

Brain Penetrant JCN068—But Not the Brain Impenetrant Erlotinib—Rapidly Decreases ¹⁸F-FDG Uptake in an Intracranial GBM

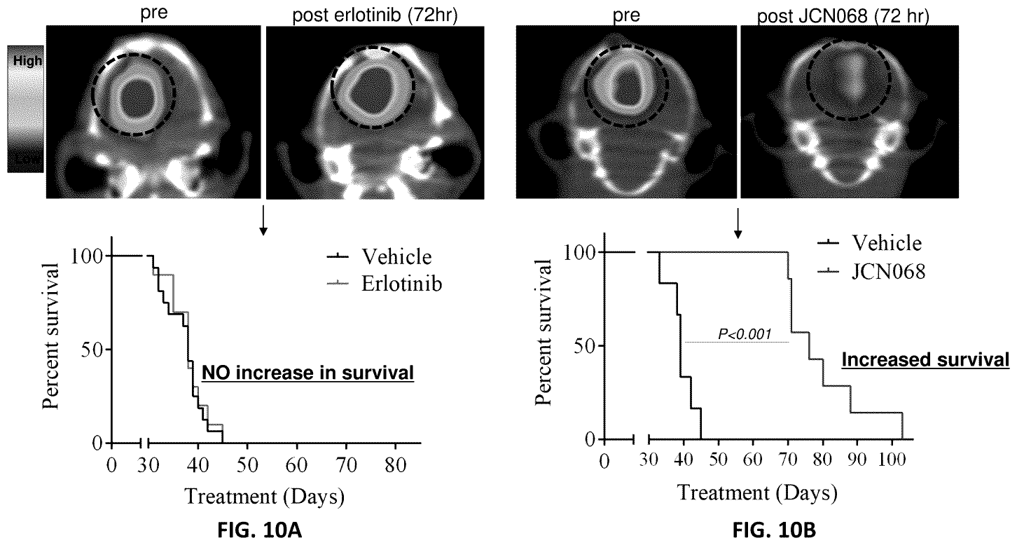

TKI's must be brain penetrant to effectively treat GBM. Many of the standard of care drugs do not have ideal brain penetrant profiles (see Table 1). To determine if the effectiveness of brain penetrant JCN068 or relatively impenetrant erlotinib can be determined by visualizing ¹⁸F-FDG uptake, mice with GBM xenografts implanted in their brains were administered either erlotinib or JCNO68. These mice were also administered ¹⁸F-FDG and PET scanned 72 hours after drug treatment. FIG. 10A shows that mice receiving erlotinib showed no reduction in ¹⁸F-FDG uptake and no increase in survival relative to mice treated with vehicle. Conversely, JCNO68 showed detectable reduction in ¹⁸F-FDG uptake and a significant increase in survival relative to mice treated with vehicle (FIG. 10B).

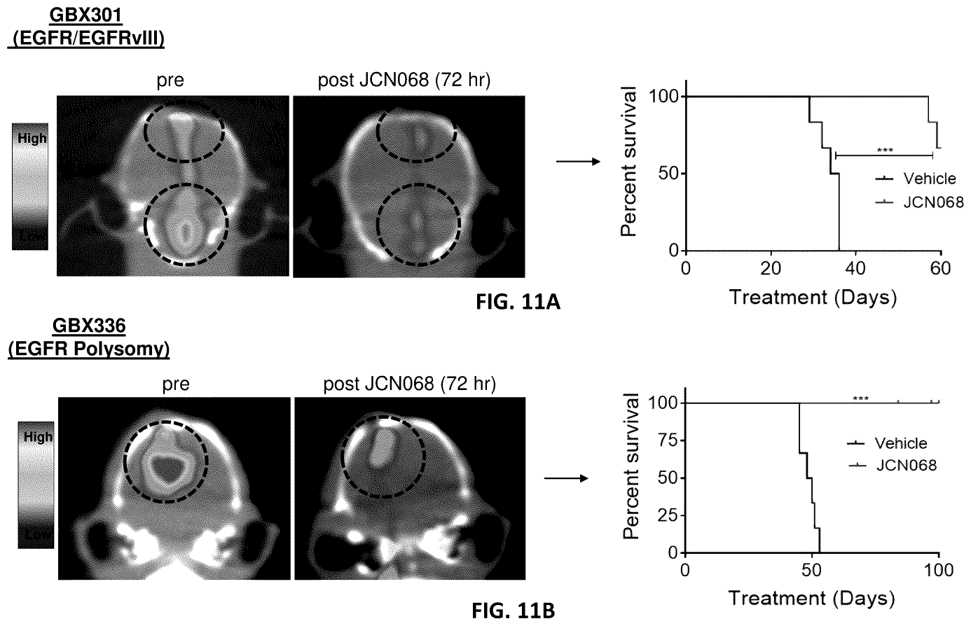

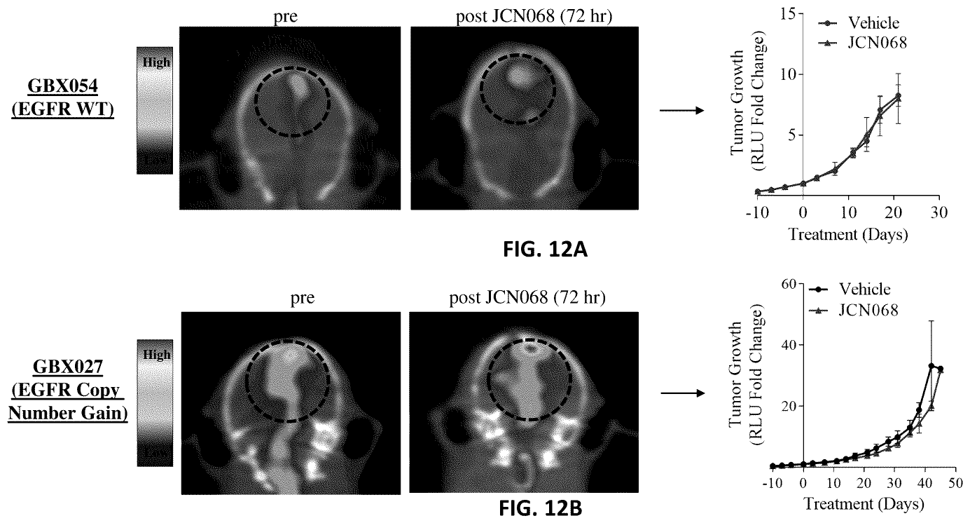

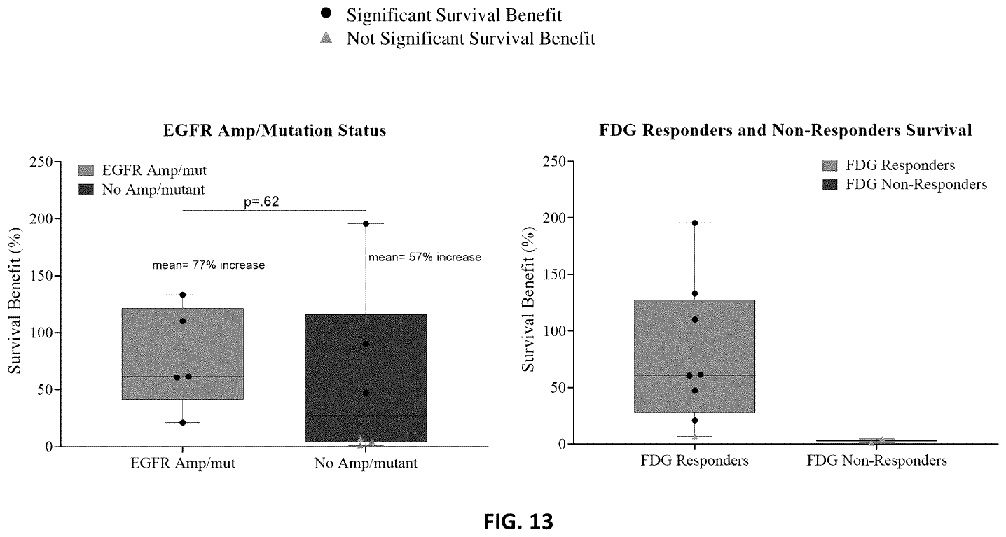

The success of JCNO68 was confirmed in the brains of mice carrying xenografts direct-from-patient orthotopic glioma xenografts that recapitulate intratumor heterogeneity. For example, xenografts GBX301 and GBX336 both showed metabolic response just 72 hours post-JCNO68 treatment, and this metabolic response predicted increased survival of the treated mice relative to control mice (FIGS. 11A and 11B). Xenografts GBX054 and GBX027 both exhibited no metabolic response 72 hours post-JCNO68 treatment, and this lack of a metabolic response predicted decreased survival of the treated mice relative to control mice (FIGS. 12A and 12B). A sizeable increase in survival benefit was observed in mice with metabolic responder xenografts relative to non-responder xenografts (FIG. 13).

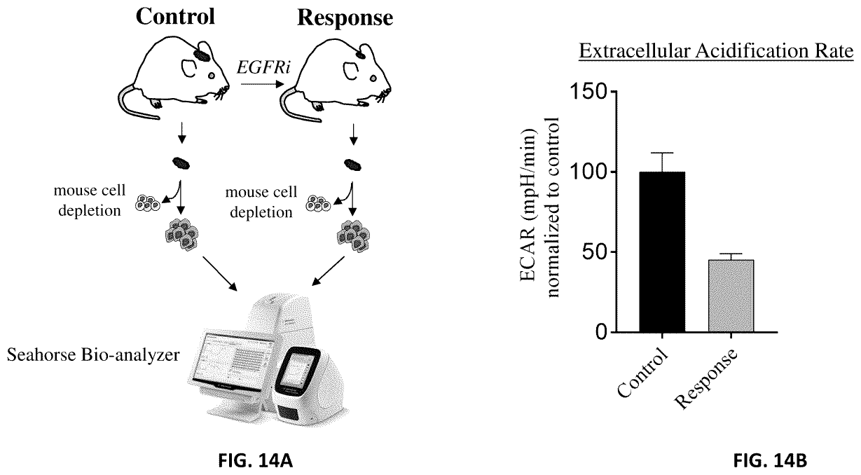

Ex Vivo Analysis of GBM Grafts
Human GBM cells were grafted into mice as described below. Cells were collected by biopsy and from untreated control mice and mice receiving treatment with an EGFR inhibitor (EGFRi) (FIG. 14A). The cells were then analyzed to determine the ECAR. FIG. 14B shows that the ECAR of mice not receiving treatment was significantly greater than the ECAR of mice that received the EGFRi.

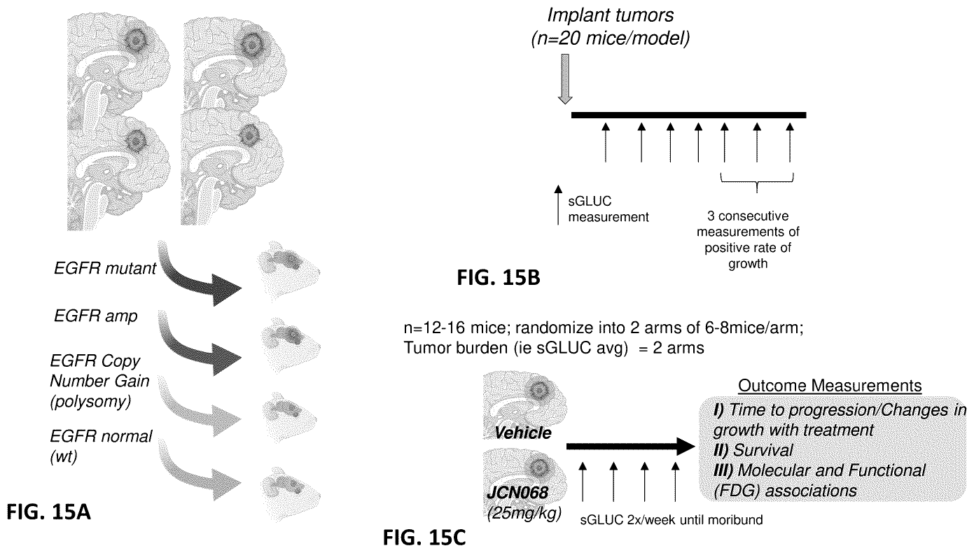

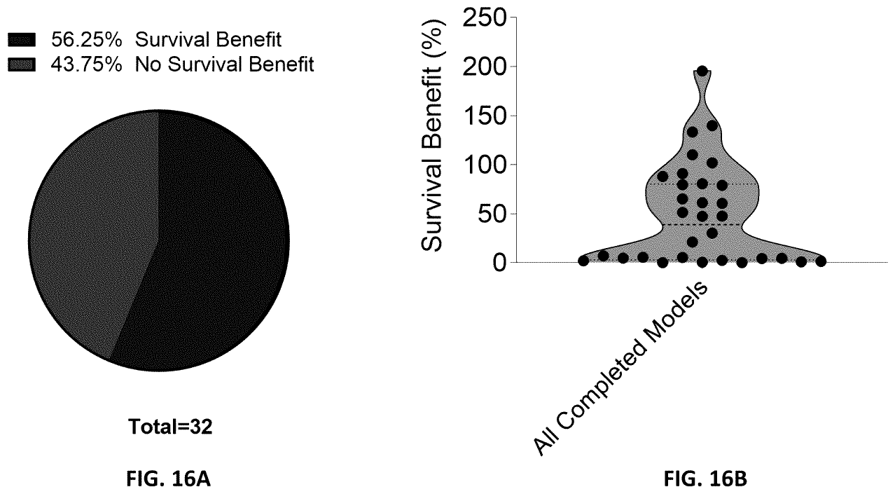

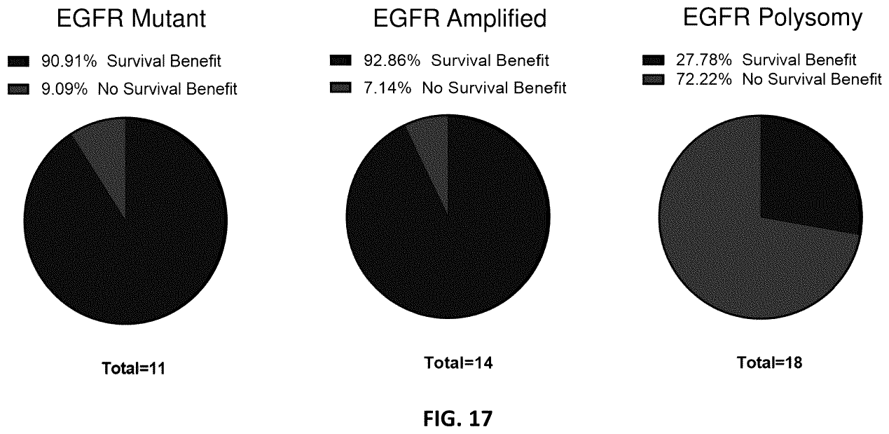

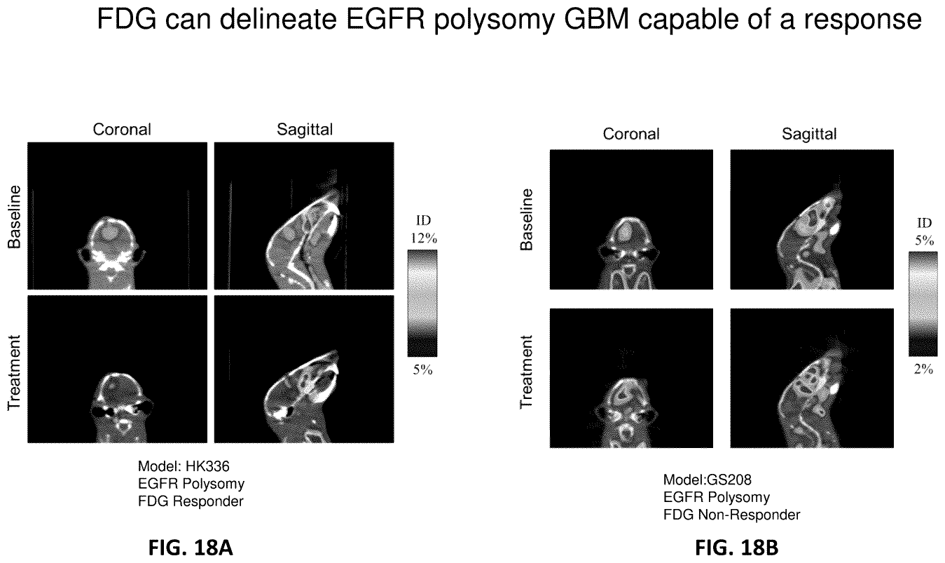

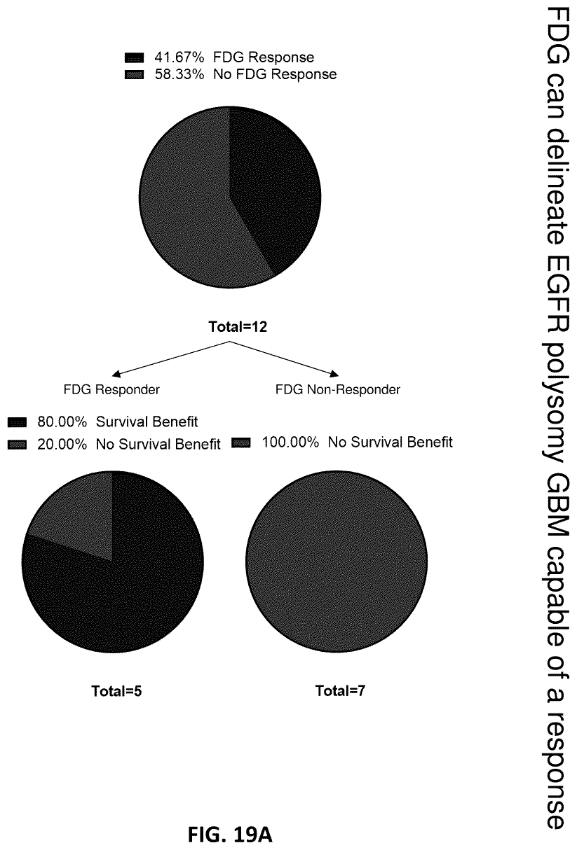

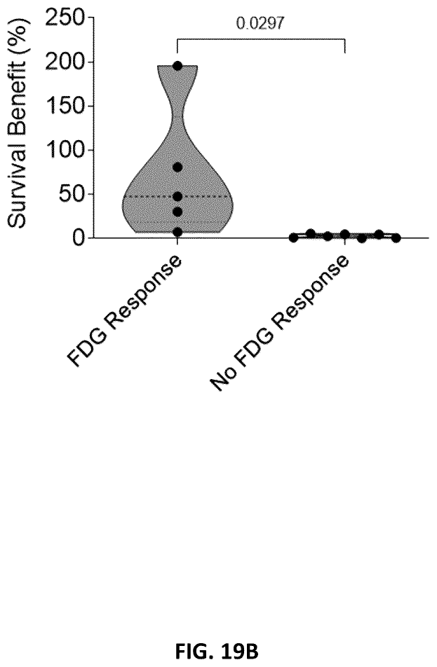

FDG Can Delineate EGFR polysomy GBM Capable of a Response
In this study, tumors from of subjects having glioblastoma with a missense or nonsense mutation in the EGFR gene, a mutation resulting in expression amplification of EGFR, or EGFR polysomy were transplanted into mice (i.e., patient derived xenografts (PDXs))as described below (FIG. 15A). A baseline measurement of plasma glucose (sGLUC) was obtained immediately prior to the tumor implantation, and after three consecutive measurements of positive tumor growth, the mice were administered 25 mg/kg JCN068 or vehicle (FIGS. 15B, 15C). Plasma glucose was measured every two weeks until the mice were moribund.
Referring to FIG. 16A, more than half of all treated mice showed a survival benefit relative to untreated mice. This survival benefit for all mice tested varied between a 50% and approximately 200% survival benefit (FIG. 16B). This variability can be at least partially attributable to the particular EGFR alteration present in the GMB tumor. For example, over 90% of the mice having a PDX derived from a glioblastoma having an EGFR genetic mutation or an EGFR amplification amplification showed over a survival benefit, while less than 30% of the mice having a PDX derived from a glioblastoma having EGFR polysomy showed a survival benefit (FIG. 17).
FDG dynamics stratify EGFR polysomy GBM patient derived xenografts capable of therapeutic response to JCNO68. Of the mice having EGFR polysomy xenographs that were identified as responders by ¹⁸F-FDG PET, 80% showed a survival benefit with JCNO68 treatment, with the survival benefit ranging from about 50% to about 200% (FIGS. 18A, 18B, 19A, 19B). None of the mice having EGFR polysomy PDXs tumors that were identified as non-responders showed a survival benefit with JCNO68 treatment (FIG. 19A).

Materials and Methods

Mice

Female NOD scid gamma (NSG), 6-8 weeks of age, were purchased from the University of California Los Angeles (UCLA) medical center animal breeding facility. Male CD-1 mice, 6-8 weeks of age, were purchased from Charles River. All mice were kept under defined flora pathogen-free conditions at the AAALAC-approved animal facility of the Division of

Laboratory Animals (DLAM) at UCLA. All animal experiments were performed with the approval of the UCLA Office of Animal Resource Oversight (OARO).

Patient-derived GBM Cells

All patient tissue to derive GBM cell cultures was obtained through explicit informed consent, using the UCLA Institutional Review Board (IRB) protocol: 10-00065. As previously described¹², primary GBM cells were established and maintained in gliomasphere conditions consisting of DMEM/F12 (Gibco), B27 (Invitrogen), Penicillin-Streptomycin (Invitrogen), and Glutamax (Invitrogen) supplemented with Heparin (5 μg/mL, Sigma), EGF (50 ng/mL, Sigma), and FGF (20 ng/mL, Sigma). All cells were grown at 37° C., 20% 02, and 5% CO₂ and were routinely monitored and tested negative for the presence of mycoplasma using a commercially available kit (MycoAlert, Lonza). At the time of experiments, most HK lines used were between 20-30 passages (exceptions HK385 p8, HK336 p15), while GS and GBM39 lines were less than 10 passages. All cells were authenticated by short-tandem repeat (STR) analysis

Reagents and Antibodies

Chemical inhibitors from the following sources were dissolved in DMSO for in vitro studies: Erlotinib (Chemietek), Nutlin-3A (Selleck Chemicals), WEHI-539 (APExBIO), Pictilisib

(Selleck Chemicals), Oligomycin (Sigma), Rotenone (Sigma). 2DG (Sigma) was dissolved freshly in media prior to usage. Antibodies used for immunoblotting were obtained from the listed sources: β-actin (Cell signaling, 3700), tubulin (Cell signaling, 3873), p-EGFR Y1086 (Thermo Fischer Scientific, 36-9700), t-EGFR (Millipore, 06-847), t-AKT (Cell Signaling, 15 4685), p-AKT T308 (Cell Signaling, 13038), p-AKT 5473 (Cell Signaling, 4060), t-ERK

(Cell Signaling, 4695), p-ERK T202/Y204 (Cell Signaling, 4370), t-S6 (Cell Signaling, 2217), p-S6 S235/236 (Cell Signaling, 4858), t-4EBP1 (Cell Signaling, 9644), p-4EBP1 S65 (Cell Signaling 9451), Glut3 (Abcam, ab15311), Glut1 (Millipore, 07-1401), p53 (Santa Cruz Biotechnology, SC-126), BAX (Cell Signaling, 5023), BIM (Cell Signaling, 2933), Bcl-2 (Cell Signaling, 2870), Bcl-xL (Cell Signaling, 2764), Mcl-1 (Cell Signaling, 5453), Cytochrome c (Cell Signaling, 4272), and Cleaved Caspase-3 (Cell Signaling, 9661). Antibodies used for immunoprecipitation were obtained from the listed sources: p53 (Cell Signaling, 12450) and Bcl-xL (Cell Signaling, 2764). Secondary antibodies were obtained from the listed sources: Anti-rabbit IgG HRP-linked (Cell Signaling, 7074) and Anti-mouse IgG HRP-linked (Cell Signaling, 7076). All immunoblotting antibodies were used at a dilution of 1:1000, except (3-actin and tubulin, which were used at 1:10,000. Immunoprecipitation antibodies were diluted according to manufacturer's instructions (1:200 for p53 and 1:100 for Bcl-xL). Secondary antibodies were used at a dilution of 1:5000.

¹⁸F-Fluorodeoxyglucose (¹⁸F-FDG) uptake assay.

Cells were plated at 5×10⁴ cells/ml and treated with designated drugs for indicated time points. Following appropriate treatment, cells were collected and resuspended in glucose-free DMEM/F12 (USBiological) containing ¹⁸F-FDG (radioactivity 1 μCi/mL). Cells were incubated at 37° C. for 1 hr and then washed three times with ice cold PBS. Radioactivity of each sample was then measured using a gamma counter.

Glucose, Glutamine, and Lactate Measurements

Cellular glucose consumption and lactate production were measured using a Nova Biomedical BioProfile Basic Analyzer. Briefly, cells were plated in 1×10⁵ cells/ml in 2 mL of gliomasphere conditions and appropriate drug conditions (n=5). 12 hrs following drug treatment, 1 ml of media was removed from each sample and analyzed in the Nova BioProfile analyzer. Measurements were normalized to cell number.

Annexin V Apoptosis Assay

Cells were collected and analyzed for Annexin V and PI staining according to manufacturer's protocol (BD Biosciences). Briefly, cells were plated at 5×10⁴ cells/ml and treated with appropriate drugs. Following indicated time points, cells were collected, trypsinized, washed with PBS, and stained with Annexin V and PI for 15 minutes. Samples were then analyzed using the BD LSRII flow cytometer.

Immunoblotting

Cells were collected and lysed in RIPA buffer (Boston BioProducts) containing Halt Protease and Phosphatase Inhibitor (Thermo Fischer Scientific). Lysates were centrifuged at 14,000×g for 15 min at 4° C. Protein samples were then boiled in NuPAGE LDS Sample Buffer (Invitrogen) and NuPAGE Sample Reducing Agent (Invitrogen) and separated using SDS-PAGE on 12% Bis-Tris gels (Invitrogen) and transferred to nitrocellulose membrane (GE

Healthcare) Immunoblotting was performed per antibody's manufacturer's specifications and as mentioned previously. Membranes were developed using the SuperSignal system (Thermo Fischer Scientific).

Immunoprecipitation

Cells were collected, washed once with PBS, and incubated in IP lysis buffer (25 mM Tris-HCL pH 7.4, 150 mM NaCl, 1 mM EDTA, 1% NP-40, 5% Glycerol) at 4° C. for 15 minutes. 300-500 μg of each sample was then pre-cleared in Protein A/G Plus Agarose Beads (Thermo Fischer Scientific) for one hour. Following pre-clear, samples were then incubated with antibody-bead conjugates overnight according to manufacturer's specifications and as mentioned previously. The samples were then centrifuged at 1000 g for 1 min, and the beads were washed with 500 μL of IP lysis buffer for five times. Proteins were eluted from the beads by boiling in 233 LDS Sample Buffer (Invitrogen) at 95° C. for 5 min. Samples analyzed by immunoblotting as previously described Immunoprecipitation antibodies were diluted according to manufacturer's instructions (1:200 for p53 and 1:100 for Bcl-xL).

GI50

The GI₅₀ Assays were performed using patient-derived glioblastoma cells. 13 concentrations at 2-fold dilutions from 40,000 nM to 9.77 nM (for GBM lines) or from 4,000 nM to 0.977 nM (for Lung Cancer lines (PC9) were plated on 384-well plates in quadruplicates with 1500 cells per well. Cells were incubated for 3 days and then proliferation was assessed by Cell Titer Glo (Promega #G7570). As a reference, Erlotinib exhibited a GI₅₀ of 642 nM (HK301) and 2788 nM (GBM39).

Mouse Xenograft Studies

For intracranial experiments, GBM39, HK336, HK393, and GS025 cells were injected (4×10⁵ cells per injection) into the right striatum of the brain of female NSG mice (6-8 weeks old). Injection coordinates were 2 mm lateral and 1 mm posterior to bregma, at a depth of 2 mm Tumor burden was monitored by secreted gaussia luciferase and following three consecutive growth measurements, mice were randomized into four treatment arms consisting of appropriate vehicles, 75 mg/kg erlotinib, 50 mg/kg Idasanutlin, or a combination of both drugs. Vehicle consisted of 0.5% methylcellulose in water, which is used to dissolve erlotinib, and a proprietary formulation obtained from Roche, which is used to dissolve Idasanutlin. Tumor burden was assessed twice per week by secreted gaussia luciferase. When possible, mice were treated for 25 days and taken off treatment and monitored for survival. Drugs were administered through oral gavage. Sample sizes were chosen based off estimates from pilot experiments and results from previous literature¹². Investigators were not blinded to group allocation or assessment of outcome. All studies were in accordance with UCLA OARO protocol guidelines.

Intracranial Delayed PET/CT Mouse Imaging

Mice were treated with indicated dose and time of erlotinib then pre-warmed, anesthetized with 2% isoflurane, and intravenously injected with 70 μCi of ¹⁸F-FDG. Following 1 hr unconscious uptake, mice were taken off anesthesia but kept warm for another 5 hr of uptake. 6 hr after the initial administration of ¹⁸F-FDG, mice were imaged using G8 PET/CT scanner (Sofie Biosciences). Per above, quantification was performed by drawing 3D regions of interest (ROI) using the AMIDE software.

Immunofluorescence

For immunofluorescence, gliomaspheres were first disassociated to single cell and adhered to the 96-well plates using Cell-Tak (Corning) according to manufacturer instructions. Adhered cells were then fixed with ice-cold methanol for 10 min then washed three times with PBS. Cells were then incubated with blocking solution containing 10% FBS and 3% BSA in PBS for 1 hr and subsequently incubated with p53 (Santa Cruz, SC-126, dilution of 1:50) antibody overnight at 4° C. The following day, cells were incubated with secondary antibody (Alexa Fluor 647, dilution 1:2000) for an hour and DAPI staining for 10 min, then imaged using a Nikon TI Eclipse microscope equipped with a Cascade II fluorescent camera (Roper Scientific). Cells were imaged with emissions at 461 nM and 647 nM and then processed using NIS-Elements AR analysis software.

DNA Sequencing

Targeted sequencing was performed for samples HK206, HK217, HK250, HK296 for the following genes BCL11A, BCL11B, BRAF, CDKN2A, CHEK2, EGFR, ERBB2, IDH1, IDH2, MSH6, NF1, PIK3CA, PIK3R1, PTEN, RB1, TP53 using Illumina Miseq. There were 1 to 2 15 million reads per sample with average coverage of 230 per gene. Copy number variants were determined for these samples using a whole genome SNP array. The genetic profile of GBM39 has been previously reported in the literature.

Whole exome sequencing was performed for samples HK157, HK229, HK248, HK250, HK254, HK296, HK301, HK336, HK350, HK390, HK393 and carried out at SeqWright. Samples were grouped into 2 pools with separate capture reactions. Nextera Rapid capture and library preparation were used and sequencing performed on a HiSeq 2500, 2×100 bp with 100× on-target coverage, 2 full rapid runs, each with 1 normal diploid control. Copy number analysis for these samples was carried out using EXCAVATOR software.

Statistical Analysis

Comparisons were made using two-tailed unpaired Student's t-tests and p values <0.05 were considered statistically significant. All data from multiple independent experiments were assumed to be of normal variance. Data represent means±s.e.m. values. All statistical analyses were calculated using Prism 6.0 (GraphPad). For all in vitro and in vivo experiments, no statistical method was used to predetermine sample size and no samples were excluded. For in vivo tumor measurements, the last data sets were used for comparisons between groups. As described above, all mice were randomized before studies.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations

We claim:

1. A method of treating a glioblastoma, the method comprising:
administering to a subject having a glioblastoma a first dose of an epidermal growth factor receptor tyrosine kinase inhibitor (EGFR TKI);
administering to the subject a detectably labeled substrate for a metabolic process;
measuring an amount of the detectably labeled substrate in the glioblastoma after administering the EGFR TKI, wherein a decrease in the amount of detectably labeled substrate relative to a reference level indicates that the glioblastoma is a metabolic responder to the inhibitor; and
if the measured amount is less than the reference level, treating the subject with the EGFR TKI for a period of time and imaging the glioblastoma to assess a change in tumor volume over the period of time.

2. A method of identifying a metabolic process inhibitor as an effective treatment for a glioblastoma, the method comprising:
administering to a subject in need thereof the metabolic process inhibitor
administering to the subject a substrate for a metabolic process, wherein the substrate is detectably labeled; and
measuring the amount of the detectably labeled substrate in the glioblastoma after administering the metabolic process inhibitor, wherein a decrease in the amount of detectably labeled substrate relative to a reference level indicates that the glioblastoma is a metabolic responder to the inhibitor; and
if the glioblastoma is identified as a metabolic responder, treating the subject with the metabolic process inhibitor for a period of time and imaging the glioblastoma to assess a change tumor volume over the period of time, wherein a decrease in tumor volume identifies the inhibitor as an effective treatment for the glioblastoma.

3. The method of claim 1 or 2, wherein the EGFR TKI or the metabolic process inhibitor is a compound having a structure of Formula I or Formula I*.

4. The method of claim 1 or 2, wherein the EGFR TKI is erlotinib, gefitinib, icotinib, afatinib, or osimertinib, or a pharmaceutically acceptable salt thereof.

5. The method of any one of claims 1-3, wherein the EGFR TKI is JCNO68.

6. The method of any one of claims 1-5, wherein the substrate is a monosaccharide, polysaccharide, amino acid, or a lipid.

7. The method of claim 6, wherein the monosaccharide is glucose.

8. The method of claim 6, wherein the monosaccharide is fluorodeoxyglucose (¹⁸F-FDG).

9. The method of any one of claims 1-8, wherein the metabolic process is glycolysis.

10. The method of any one of claims 1-9, wherein administering the detectably labeled substrate precedes administering the EGFR TKI or the metabolic process inhibitor.

11. The method of any one of claims 1-9, wherein administering the EGFR TKI or the metabolic process inhibitor precedes administering the detectably labeled substrate.

12. The method of any one of claims 1-10, wherein the reference level is determined by measuring the uptake of the detectably labeled substrate in the glioblastoma before administering the EGFR TKI.

13. The method of any one of claims 1-12, wherein measuring the amount of labeled substrate in the glioblastoma comprises performing a positron emission tomography PET scan.

14. The method of any one of claims 1-13, wherein the reference level is a known level representative of typical glucose uptake in a glioblastoma.

15. The method of any one of claims 1-14, wherein the imaging comprises at least a first imaging between 0 to about 7 days after treating the subject with the EGFR TKI or the metabolic inhibitor.

16. The method of any one of claims 1-15, wherein the imaging comprises a second imaging between about 7 days and about 30 days after treating the subject with the EGFR-TKI or metabolic process inhibitor.

17. A method of treating a glioblastoma, the method comprising:
administering to a subject having a glioblastoma a first dose of an epidermal growth factor receptor tyrosine kinase inhibitor (EGFR TKI);
administering to the subject a substrate for a metabolic process, wherein the substrate has an exchangeable proton;
detecting a pH change in the glioblastoma after administering the EGFR TKI, wherein an increase in the pH relative to a reference level indicates that the glioblastoma is a metabolic responder to the inhibitor; and
if the pH is greater than the reference level, treating the subject with the EGFR TKI for a period of time and imaging the glioblastoma to assess a change in tumor volume over the period of time.

18. A method of identifying a metabolic process inhibitor as an effective treatment for a glioblastoma, the method comprising:
administering to a subject in need thereof the metabolic process inhibitor administering to the subject a substrate for a metabolic process; and
measuring the pH in the glioblastoma after administering the metabolic process inhibitor, wherein an increase in the pH relative to a reference level indicates that the glioblastoma is a metabolic responder to the inhibitor; and
if the glioblastoma is identified as a metabolic responder, treating the subject with the metabolic process inhibitor for a period of time and imaging the glioblastoma to assess a change tumor volume over the period of time, wherein a decrease in tumor volume identifies the inhibitor as an effective treatment for the glioblastoma.

19. The method of any one of claim 17 or 18, wherein the imaging of the glioblastoma is by chemical exchange saturation transfer (CEST) magnetic resonance imaging (MRI).

20. The method of any one of claims 1-19, wherein the glioblastoma comprises a wild type or mutant EGFR, a mutation that results in increased EGFR expression relative to a normal control, or an EGFR with an altered copy number.

21. The method of claim 20, wherein the altered copy number of EGFR is caused by amplification of the EGFR or by polysomy.

22. The method of claim 21, wherein the polysomy is trisomy 7.

23. The method of any one of claims 1-22, further comprising administering an additional therapy to the subject if the glioblastoma is identified as a metabolic responder, or if the glioblastoma does not decrease in volume over the period of time.

24. The method of claim 23, wherein administering the additional therapy commences subsequent to identifying the glioblastoma as a metabolic responder or if the glioblastoma does not decrease in volume over the period of time.

25. The method of claim 24, wherein the additional therapy is a cytoplasmic p53 stabilizer.

26. The method of claim 25, wherein the cytoplasmic p53 stabilizer is an MDM2 inhibitor.

27. The method of claim 26, wherein the MDM2 inhibitor is nutlin RO5045337, RO5503781, RO6839921, SAR405838, DS-3032, DS-3032b, or AMG-232.

28. The method of claim 26, wherein the cytoplasmic p53 stabilizer is a BCL-2 inhibitor.

29. The method of claim 28, wherein the BCL-2 inhibitor is antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, or APG-1252.

30. The method of claim 25, wherein the cytoplasmic p53 stabilizer is a Bcl-xL inhibitor.

31. The method of claim 30, wherein the Bcl-xL inhibitor is WEHI 539, ABT-263, ABT-199, ABT-737, ABBV-155, sabutoclax, AT101, TW-37, APG-1252, or gambogic acid.

32. The method of any one of claims 25-31, wherein the EGFR TKI or the metabolic process inhibitor and the cytoplasmic p53 stabilizer are administered conjointly.

33. A method of monitoring a glioblastoma tumor in a subject receiving an EGFR TKI therapy, the method comprising:
assessing the size or volume of the glioblastoma tumor at a first time point;
assessing the size or volume of the glioblastoma tumor at at least one subsequent time point, wherein the patient has received at least one dose of the therapy between the first and the at least one subsequent time point; and
comparing the sizes or volumes of the glioblastoma tumor at the first time point and at the at least one subsequent time point, wherein a decrease in tumor size or volume at a subsequent time point relative to the first time point indicates the therapy is effective.

34. The use of an EGFR tyrosine kinase inhibitor (TKI) in the manufacture of a medicament for the treatment of a glioblastoma tumor according to a method wherein the effectiveness of the treatment is determined by:
a) assessing the size or volume of the glioblastoma tumor at a first time point;
b) assessing the size or volume of the glioblastoma tumor at at least one subsequent time point; and
c) comparing the sizes or volumes of the glioblastoma tumor at the first time point and at the at least one subsequent time point, wherein a decrease in tumor size or volume at a subsequent time point relative to the first time point indicates the treatment is effective.

35. Use of an EGFR tyrosine kinase inhibitor (TKI) for the treatment of a glioblastoma tumor according to a method that comprises:
a) assessing the size or volume of the glioblastoma tumor at a first time point during the treatment;
b) assessing the size or volume of the glioblastoma tumor at at least one subsequent time point during the treatment; and
c) comparing the sizes or volumes of the glioblastoma tumor at the first time point and the at least one subsequent time point, wherein a decrease in tumor size or volume at a subsequent time point relative to the first time point indicates the treatment is effective.

36. The use of claim 34 or 35, wherein the glioblastoma tumor comprises an EGFR polysomy.

37. The use of claim 34 or 35, further comprising determining or identifying an EGFR polysomy in the glioblastoma.

38. The use of claim 36, wherein the EGFR polysomy is trisomy 7.

39. The use of any one of claims 34-38, wherein the glioblastoma tumor is assessed for responsiveness to the EGFR TKI.

40. The use of claim 39, wherein the responsiveness to the EGFR TKI is assessed by:
administering to a subject in need thereof the metabolic process inhibitor administering to the subject a substrate for a metabolic process, wherein the substrate is detectably labeled; and
measuring the amount of the detectably labeled substrate in the glioblastoma after administering the metabolic process inhibitor, wherein a decrease in the amount of detectably labeled substrate relative to a reference level indicates that the glioblastoma is a metabolic responder to the inhibitor.

41. The use of claim 40, wherein the detectably labeled substrate is fluorodeoxyglucose (¹⁸F-FDG).

42. The use of claim 40 or 41, wherein measuring comprises scanning the glioblastoma with positron emission tomography.

43. A method of treating a glioblastoma, the method comprising:
identifying a polysomy in a glioblastoma tumor;
administering to a subject having a glioblastoma a first dose of an epidermal growth factor receptor tyrosine kinase inhibitor (EGFR TKI);
administering to the subject a detectably labeled substrate for a metabolic process;
measuring an amount of the detectably labeled substrate in the glioblastoma after administering the EGFR TKI, wherein a decrease in the amount of detectably labeled substrate relative to a reference level indicates that the glioblastoma is a metabolic responder to the inhibitor; and
if the measured amount is less than the reference level, treating the subject with the EGFR TKI for a period of time and imaging the glioblastoma to assess a change in tumor volume over the period of time.

44. The method of claim 43, wherein the polysomy is trisomy 7.

45. The method of claim 43 or 44, wherein the EGFR TKI is JCN068, erlotinib, gefitinib, icotinib, afatinib, or osimertinib, or a pharmaceutically acceptable salt thereof.

46. The method of claim 43, wherein the detectably labeled substrate is fluorodeoxyglucose (¹⁸F-FDG).

47. The method of claim 43, wherein measuring comprises scanning the glioblastoma with positron emission tomography.
